(19) United States
(12) Patent Application Publication
(10) Pub. No.: US 2019/0055608 A1
(43) Pub. Date: Feb. 21, 2019

(54) MULTI-MODAL PROSTATE CANCER MARKER

(71) Applicants: ONTARIO INSTITUTE FOR CANCER RESEARCH (OICR), Toronto (CA); UNIVERSITY HEALTH NETWORK, Toronto (CA)

(72) Inventors: Paul Boutros, Toronto (CA); Robert G. Bristow, Toronto (CA); Sylvia Shiah, Toronto (CA); Michael Fraser, Toronto (CA); Veronica Sabelnykova, Toronto (CA); Vincent Huang, Toronto (CA); Lawrence Heisler, Toronto (CA); Takafumi Yamaguchi, Toronto (CA); Julie Livingstone, Toronto (CA)

(21) Appl. No.: 16/074,635

(22) PCT Filed: Feb. 2, 2017
(86) PCT No.: PCT/CA2017/000023

§ 371 Date: Aug. 1, 2018

Related U.S. Application Data
(60) Provisional application No. 62/290,246, filed on Feb. 2, 2016.

(51) Int. Cl. C12Q 1/6886; G06F 19/20; G06F 19/18

(52) U.S. Cl. CPC C12Q 1/6886; G06F 19/20; G06F 19/18; C12Q 2600/158; C12Q 2600/118; C12Q 2600/154; C12Q 2600/156; C12Q 2600/112

(57) ABSTRACT

There is described herein a method of prognosing and/or predicting disease progression in subject with prostate cancer, the method comprising: a) providing a sample containing genetic material from cancer cells; b)determining or measuring at least 2 patient biomarkers regarding the prostate cancer tumor selected from the group consisting of: T category, ACTL6B methylation, TCERGL1 methylation, chr7:61 Mbp inter-chromosomal translocation, ATM single nucleotide variants and MYC copy number aberrations; c) comparing said patient biomarkers to corresponding reference or control biomarkers; and d) determining the likelihood of disease progression; wherein a likelihood of disease progression is higher with each of ACTL6B hyper-methylation, TCERGL1 hypo-methylation, higher T category, and higher incidences of chr7:61 Mbp inter-chromosomal translocation, ATM single nucleotide variants and MYC copy number aberrations, when the difference is statistically significant on comparison with the reference or control biomarkers.

### CROSS REFERENCE TO RELATED APPLICATIONS

This application claims the benefit of priority of U.S. Provisional Patent Application No. 62/290,246 filed Feb. 2, 2016 and incorporated herein by reference in its entirety.

### FIELD OF INVENTION

The present disclosure relates generally to a prostate cancer biomarker signature. More particularly, the present disclosure relates to a multi-model signature for the prognosis of prostate cancer outcomes, which can inform treatment decisions and guide therapy.

### BACKGROUND

Prostate cancer is the most commonly diagnosed non-skin malignancy in men, resulting in an estimated 256,000 deaths worldwide in 2010¹. While the vast majority of men present with localized, and thus potentially curable disease, current clinical prognostic factors explain only a fraction of the heterogeneity of treatment response. These factors thus do not optimally triage individual patients into appropriate risk groupings that determine the appropriate treatment aggression^{2,3}.

Localized prostate cancers exhibit striking inter-tumoural heterogeneity, at both the genomic^{4,5} and microenvironmental levels⁶. In particular, intermediate risk prostate cancers are localized, non-indolent and clinically heterogeneous. Despite current management with either surgery or radiotherapy, more than 30% of men suffer relapse of their disease; in 10% of these men (approximately 10,000 a year), rapid biochemical recurrence can portend prostate cancer-specific death⁷. Having a rigorous understanding of the genetic factors driving progression and aggression in the initial pre- and post-treatment setting is a critical need for both clinicians and genetic researchers, as distinct genomic pathways of progression could define prostate cancer sub-types leading to novel curative therapeutics. It is of utmost importance to be able to identify the genetic drivers of localized, non-indolent prostate cancer, as they cannot be inferred from genomic studies of metastatic castrate-resistant prostate cancer (mCRPC), due to tumour cell selection and adaption to secondary androgen deprivation therapy⁸.

### SUMMARY OF INVENTION

In a general aspect, there is provided a method of prognosing and/or predicting disease progression in subject with prostate cancer. The method comprises use of at least 2 patient biomarkers determined or measured from genetic material of cancer cells and comparing them to corresponding reference or control measures of the same biomarkers. The biomarkers are selected from T category, and aberrations in ACTL6B, TCERGL1, chr7:61 Mbp, ATM and MYC. Statistically significant aberrations of the subject biomarkers when compared to the reference biomarkers would be indicative of a worse outcome.

In an aspect, there is provided a method of prognosing and/or predicting disease progression in subject with prostate cancer, the method comprising: a) providing a sample containing genetic material from cancer cells; b)determining or measuring at least 2 patient biomarkers regarding the prostate cancer tumor selected from the group consisting of: T category, ACTL6B methylation, TCERGL1 methylation, chr7:61 Mbp inter-chromosomal translocation, ATM single nucleotide variants and MYC copy number aberrations; c) comparing said patient biomarkers to corresponding reference or control biomarkers; and d) determining the likelihood of disease progression; wherein a likelihood of disease progression is higher with each of ACTL6B hyper-methylation, TCERGLI hypo-methylation, higher T category, and higher incidences of chr7:61 Mbp inter-chromosomal translocation, ATM single nucleotide variants and MYC copy number aberrations, when the difference is statistically significant on comparison with the reference or control biomarkers.

In an aspect, there is provided a computer-implemented method of predicting disease progression in patient with prostate cancer, the method comprising: a) receiving, at at least one processor, data reflecting at least 2 patient biomarkers regarding the prostate cancer tumor selected from the group consisting of: T category, ACTL6B methylation, TCERGL1 methylation, chr7:61 Mbp inter-chromosomal translocation, ATM single nucleotide variants and MYC copy number aberrations; b) constructing, at the at least one processor, an expression profile corresponding to the expression levels; c) comparing, at the at least one processor, said patient biomarkers to corresponding reference or control biomarkers; d) determining, at the at least one processor, the likelihood of disease progression; wherein a likelihood of disease progression is higher with each of ACTL6B hyper-methylation, TCERGLI hypo-methylation, higher T category, and higher incidences of chr7:61 Mbp inter-chromosomal translocation, ATM single nucleotide variants and MYC copy number aberrations, when the difference is statistically significant on comparison with the reference or control biomarkers.

In an aspect, there is provided a computer program product for use in conjunction with a general-purpose computer having a processor and a memory connected to the processor, the computer program product comprising a computer readable storage medium having a computer mechanism encoded thereon, wherein the computer program mechanism may be loaded into the memory of the computer and cause the computer to carry out the method described herein.

In an aspect, there is provided a computer readable medium having stored thereon a data structure for storing the computer program product described herein.

In an aspect, there is provided a device for predicting disease progression in patient with prostate cancer, the device comprising: at least one processor; and electronic memory in communication with the at one processor, the electronic memory storing processor-executable code that, when executed at the at least one processor, causes the at least one processor to: a) receive data reflecting at least 2 patient biomarkers regarding the prostate cancer tumor selected from the group consisting of: T category, ACTL6B methylation, TCERGL1 methylation, chr7:61 Mbp inter-chromosomal translocation, ATM single nucleotide variants and MYC copy number aberrations; b) compare said patient biomarkers to corresponding reference or control biomarkers; and c) determining, at the at least one processor, the likelihood of disease progression; wherein a likelihood of disease progression is higher with each of ACTL6B hyper-methylation, TCERGL1 hypo-methylation, higher T category, and higher incidences of chr7:61 Mbp inter-chromosomal translocation, ATM single nucleotide variants and MYC copy number aberrations, when the difference is statistically significant on comparison with the reference or control biomarkers.

Other aspects and features of the present disclosure will become apparent to those ordinarily skilled in the art upon review of the following description of specific embodiments in conjunction with the accompanying figures.

### BRIEF DESCRIPTION OF FIGURES

Embodiments of the present disclosure will now be described, by way of example only, with reference to the attached Figures.

FIG. 1 shows Global Mutational Profile of Localized Non-indolent Prostate Cancer. We comprehensively analyzed the genomic profiles of 200 localized, non-indolent prostate tumours. a) Each column represents an individual prostate tumour with whole-genome sequencing, sorted first by Gleason score (GS), then by the number of somatic single nucleotide variants identified (‘SNV’; top). The middle and bottom panels give the number of genomic rearrangements (‘GR’; middle) and copy number aberrations (‘CNA’; bottom). The clinical covariates of Gleason score, PSA, T category, and age are shown, with a colour key for each. The boxplots to the right of each panel show the association between total mutation load and GS, along with P values from one-way ANOVAs. b) Correlation between mutation load (PGA=percent genome altered; INV=inversions; CTX=inter-chromosomal translocations; SNVs) and clinical variables. Background shading indicates Bonferroni-adjusted P values; size and colour of dots show Spearman's correlation.

FIG. 2 shows Coding Somatic SNVs are Rare in Non-indolent, Localized Tumours. We created a consistent and standardized set of somatic SNVs predictions in the exome from a set of 477 tumours. Tumours are sorted by GS (bottom covariates), then by the total number of coding SNVs identified per sample (top barplot). The proportion of each type of base change is given in the middle barplot, and the heatmap displays the 19 most recurrently mutated genes, each found in at least 6 samples, ranked by the number of somatic SNVs.

FIG. 3 shows Recurrent Kataegis and Chromothripsis in Prostate Cancer. We assessed the frequency and consequences of two localized hypermutation phenomena in Gleason score 3+3, 3+4 and 4+3 tumours: chromothripsis and kataegis. a) For each tumour we quantified the extent of chromothripsis using ShatterProof and ranked samples in descending order of evidence of a chromothriptic event (top barplot). We next explored the association of chromothripsis with some measures of mutational burden, known prostate cancer genes with recurrent CNAs, novel chromothripsis associated genes, TMPRSS2:ERG fusion status, known prostate cancer genes with recurrent SNVs and clinical variables. The barplots to the right give statistical-significance of each association, (Mann-Whitney U-test for genes, Kendall's Tau for clinical covariates). b) We quantified the presence of kataegic events and visualized them as in part a) above, this time using the tests of proportions. The top barplot shows the score of the strongest kataegic event.

FIG. 4 shows Multi-Modal Prediction of Disease Relapse. a) We defined 40 properties of prostate cancers, including mutation density, presence/absence of chromothripsis and kataegis, CNAs and a series of recurrent somatic mutations. For each of these, we calculated the association with biochemical recurrence (BCR) using a CoxPH model and report the hazard ratio (HR), 95% confidence interval and P value (Wald test). b) Kaplan-Meier (K-M) plot of ATM nonsynonymous SNVs. c) K-M plot of TCERG1L for the 5′ and 3′ probes of the gene body. d) ROC curves for a multi-modal biomarker predicting biochemical recurrence, tested via cross-validation (yellow) and a PGA marker (green). The blue dots represent the operating point (maximum balanced accuracy).

FIG. 5 shows Study design. The overall study cohort consists of 137 patients who underwent radical prostatectomy (‘surgery’) and 147 patients who underwent image-guided radiotherapy for localized prostate cancer (‘biopsy’). For surgery patients, a fresh frozen tissue specimen from the index lesion was obtained for macro-dissection. For radiotherapy patients, a fresh frozen needle core ultrasound-guided biopsy to the index lesion was obtained for macro-dissection. All 284 tumour DNA specimens were analyzed for copy number aberrations (CNA) by OncoScan SNP arrays. Of these tumour DNA specimens, 130 were selected for further analysis by whole-genome sequencing (as there was a matched normal DNA specimen from whole blood). For a subset of analyses, additional data (numbers as indicated) from publicly available whole-genome or whole-exome sequencing datasets were re-aligned, re-analysed, and integrated to maximize statistical power.

FIG. 6 shows Comparison of molecular aberrations. a) Pairwise comparison scatterplot of data type as indicated on the x- and y-axes. Spearman correlation and unadjusted P values are provided. b) Scatterplots and boxplots of each mutation burden (CNA, CTX, INV, SNV counts and PGA) vs. clinical variables (age, Gleason score, T category, PSA and ETS consensus) is provided along with a model derived P value, as described in methods. Dots represent values for individual samples.

FIG. 7 shows Non-coding SNV profile. We analyzed 70 non-coding recurrent somatic SNVs: defined as at least 2% (4/200) of tumours having mutations in the same, non-coding position. a) The central heatmap shows the 70 recurrent ncSNVs (rows) and the samples they are present in (columns), with colour indicating their variant allele frequency (VAF). The top barplot indicates the total number of ncSNVs mutated in each sample, while the right barplot gives the total number of samples in which a ncSNV is mutated. b) Boxplot showing VAF for recurrent ncSNVs. Each dot indicates the VAF of a recurrent ncSNV for a sample. The recurrent ncSNVs (rows) were sorted by median VAF. c) TFBS bias in aberrations. To determine if ncSNVs were biased towards specific transcription-factor binding sites (TFBSs), we tested if experimentally-derived TFBS locations from ENCODE were enriched for aberrations of different types using the binomial test. Heatmap of 58 TFBS cell lines for each sample coloured by the data type or combination of data types (SNV, CNV, and CTX flanked by 10 kbp) if it is aberrant in more samples than expected by chance (Binomial test with FDR adjusted P value). The samples are ordered by the number of significantly aberrant TFBSs (top barplot), the TFBS cell lines are ordered by fraction of samples with significantly mutated TFBSs by cell line (right barplot), covariates of pathologic Gleason score, PSA, T category, and patient age are displayed at the bottom. d) Predicted chromatin effects of recurrent ncSNVs. The left heatmap shows E-values, which measure the expected proportion of SNPs (found in the 1000 Genomes Project) with a larger predicted effect for a chromatin feature, predicted by DeepSEA. The right heatmap shows the overlaps between chromatin elements detected by LNCaP ChIP-seq experiments and ncSNVs. The FDR adjusted P values (Q values) for the DeepSEA or ChiP-seq experiment features are shown above each plot. The ncSNVs Q values for DeepSEA and ncSNV recurrence are shown on the right. Experimental conditions (cell line type, chromatin feature, and treatment) of the ChIP-seq data are represented by the covariates at the bottom. The heatmaps and barplots were sorted by Q values.

FIG. 8 shows Genome rearrangements overview. a) Global overview of somatic structural variants in 180 localized Gleason score 3+3, 3+4 and 4+3 prostate cancers. The central heatmap shows per-sample inter-chromosomal translocations (CTXs), inversions and deletions for 1 Mbp bins across the genome (columns) and for each patient (rows). The striking TMPRSS2-ERG peak on chromosome 21 is by far the most frequent aberration, but additional recurrent inversion breakpoints were identified on chromosome 3 and 10, and CTX breakpoints on chromosome 6. b) Number of CTXs joining each chromosome pair and their occurrences relative to random chance. Dot size represents the number of translocations enriched (number greater than expected) while background colour indicates their significance as calculated using a one-tailed permutation test (1 million replicates) with FDR correction. c) Mean shortest distance between a CTX and the corresponding nearest HiC point in each chromosome pair. Dot size represents the difference between the mean observed CTX-HiC distances and their expected distances, while the background indicates significance as calculated using a one-tailed permutation test (1 million replicates) corrected using the FDR method. Orange dots indicate distances greater than expected by chance alone (upper-right), while blue dots show distances smaller than expected by chance alone (lower-left).

FIG. 9 shows Effects of inversion on mRNA abundance and PTEN. a) For each gene in the inversion window (chr10:89-90 Mbp), mRNA abundance levels were re-normalized and centered by the median across all patients. Boxplot (top) demonstrates the renormalized mRNA abundance levels (y-axis) of patients with no inversion (n=70, orange) and inversions (n=3, green) for each gene. A linear model was used to calculate the P values between the two patient groups. Barplot (bottom) shows unadjusted P values with gene ordered based on chromosome location. b) Spearman's p was used to identify the top ten genes most correlated with PTEN mRNA abundances. The per sample mean mRNA abundances of the ten genes was used to represent the overall effects of various types of PTEN inactivation. PTEN inactivation as a result of CNV loss led to a significantly lower abundance of PTEN associated proteins when compared to copy number neutral PTEN via the Mann-Whitney U-test (p=2.0×10-4) whereas PTEN inversions yielded further reduced abundances (p=0.016). c) For each gene in the inversion window (chr3:129-130 Mbp), mRNA abundance levels were re-normalized and centered by the median across all patients. Boxplot (top) shows the renormalized mRNA abundance levels (y-axis) of patients with no inversion (n=65, orange) or inversions (n=8, green) for each gene. A linear model was used to calculate P values between the two patient groups. Barplot (bottom) shows the P values with genes ordered based on chromosome location.

FIG. 10 shows Hypermutation associations. a) Boxplot of ShatterProof scores grouped by T category. Each grey dot represents a single sample. P value is from a one-way ANOVA. b) To assess the association between genome stability (measured as PGA) and the presence of one or more chromothriptic events in a tumour, we compared the mean PGA between tumours with a chromothriptic event (4.28%±5.04%) and those without one (7.79%±5.3%). This difference of 3.52% was statistically significant (p=1.10×10-3; two-sided t-test). c) To assess the association between genome stability (measured as PGA) and the presence of one or more kataegic events in a tumour, we compared the mean PGA between tumours with a kataegic event (6.87%±5.62%) and those without one (4.34%±5.13%). This difference of 2.53% was statistically significant (p=7.52×10-3; two-sided t-test). d) Scatterplot of ShatterProof scores vs. percent of infiltrating immune cells as measured by a pathologist. e) Scatterplot of ShatterProof scores vs. estimated immune score calculated by the ‘Estimate of STromal and Immune cells in Malignant Tumours’ (ESTIMATE) software. For both these plots, Spearman's p is given, along with its P value. f) Scatterplot showing the correlation between pathologist and ESTIMATE predictions on 22 samples.

FIG. 11 shows Chromothripsis associations and mutational burden. a) Scatterplots of mRNA abundance vs. ShatterProof scores for four genes found to be associated with chromothripsis. Spearman's p are displayed, along with their respective P values. Boxplots of mRNA abundance vs. copy number status (DEL=deletion, NEU=copy number neutral). P values are from two-sided t-tests. b) Scatterplots of mutation burden (SNV, INV, CNA, CTX counts) and qpure cellularity values against ShatterProof score. Spearman's p and corresponding P values are shown.

FIG. 12 shows Characteristic of mRNA genes and methylation probes in chromothriptic regions. a) Histogram of percentiles from mRNA genes (2,197 unique genes) located in a chromothriptic region. Upper left corner indicates Pearson's correlation between each bin and the frequency of genes that reside in that bin. b) A histogram as in a) for the 43,985 unique methylation probes located in chromothriptic regions. c) Boxplot of genes that are in chromothriptic regions vs. genes not in chromothriptic regions and which are deleted in at least one patient. Only non-chromothriptic patients are included, making this analysis conservative. P value was from a two-sided Wilcoxon rank-sum test.

FIG. 13 shows mRNA-methylation associations in tumours with focal genomic events. a) Density plot of Spearman correlations between the 10,000 most variable methylation probes and the 10,000 most variable mRNA transcripts in tumours with chromothriptic events, kataegic events, and neither focal abnormality. b) Density plot as in a) for the 14,778 methylation probes in promoter regions and their corresponding mRNA transcripts. c) Scatterplot of methylation (β-values for cg07227024, on chr2q) and mRNA abundance for OR2AK2 (on chrlq), which have the highest difference in correlations between chromothriptic (R=−0.90, p=9.42×10-6) and non-chromothriptic (R=0.52, p=2.0×10-4) tumours. Dotted lines represent the regression line for each group. d) Enrichment pathway network plot of genes differentially correlated between chromothriptic and stable samples in promoter regions (|δ|>0.8). Each node represents a gene set, defined as the set of genes which underlie a functional profile by g:Profiler. Node size corresponds to the number of genes within the gene set. Colour of the node represents the significance of the enriched gene set (hypergeometric test) ranging from FDR adjusted P values: 1.99×10-3 to 0.05 (red to pink). Gene sets are connected by a grey line if they share common genes while the thickness of the line corresponds to the size of the overlap. Gene sets with similar functions are grouped together by a purple dotted circle.

FIG. 14 shows Methylation survival validation. Kaplan-Meier plots of the six prognostic methylation probes in the validation dataset (100 prostate tumours). Statistical analyses were done via CoxPH modeling and P values were generated by the Wald test, except for a) where the log-rank test was performed due to failure of the proportional-hazards assumption. a) TCERG1L-3′. b) SOX14. c) TUBA3C. d) TCERG1L-5′. e) MIR129-2. f) ACTL6B.

FIG. 15 shows Multi-modal signature survival. a) A Kaplan-Meier plot for a multi-modal biomarker predicting biochemical recurrence, tested via cross-validation. This curve shows prediction of 18-month biochemical relapse-free survival. b) A Kaplan-Meier plot of the same biomarker, showing full biochemical relapse-free survival to the maximum follow-up time. In both plots, P values are generated using the Wald test.

FIG. 16 shows Copy-number aberrations in localized prostate cancer. To explore the landscape of CNAs in localized prostate cancer we generated genome-wide profiles for 284 tumours using Affymetrix OncoScan arrays. a) The resulting CNA profiles were clustered using Jaccard similarity and Ward's distance, identifying six distinct patient groupings, including a copy-number quiet group (Cluster 4). Red indicates copy number gain while blue indicates copy number loss. Each row represents a sample, and each column represents a gene. b) Genomic instability PGA (assessed as the Percentage of the Genome with a CNA) increases with Gleason score (p=2.97×10⁻⁶; one-way ANOVA). c) Nevertheless, no individual gene is mutated at statistically different frequencies between GS 3+3, 3+4, and 4+3 patients, as shown by the proportion of alterations for each GS. Genes are ordered by genomic coordinates per chromosome. d) GISTIC2.0 was used to identify recurrently aberrant regions in 284 samples. Each row represents a sample and each column represents a statistically significant GISTIC peak, with a representative gene selected from each. Samples are clustered using Jaccard similarity and Ward's distance measure. The panel on the left shows copy number alterations in genes commonly altered in prostate cancer.

FIG. 17 shows Consensus clusters of CNAs. a) Consensus cluster heatmap showing how often samples cluster together. Rows and columns are both samples. Colour shading from white to blue indicates consensus values ranging from 0-1, where 0 indicates samples never cluster together and 1 indicates that samples always cluster together. The covariate bar at the top designates which cluster the sample was assigned to. b) Delta area plot shows the relative change in area under the cumulative distribution function (CDF) curve as the number of clusters (k) increase. c) Heatmap of 284 copy number aberration profiles from OncoScan FFPE Express 2.0 and 3.0 arrays, after being mapped to gene annotation. Gains are shown in red, deletions shown in blue, and copy neutral in white. Each column is a gene, ordered by genomic co-ordinate, and each row is a patient grouped by Gleason score and Lalonde subtype³ and ordered by percent genome altered (PGA). Known prostate cancer genes are labelled at the top of the plot and chromosome boundaries are marked by the colour bar at the top. Profiles are annotated with Gleason score, PSA, T category, age at treatment, Lalonde cluster assignment and our clusters (CPC-GENE cluster, from FIG. 16).

FIG. 18 shows Clinical vs. PGA and CNA clusters. a) Scatterplot showing correlation between percent genome altered and patient age. Pearson (R) and Spearman (p) correlation values are displayed, with their respective P values. b) Box plot comparing percent genome altered across T categories, with the one-way ANOVA P value. Grey dots represent values for individual samples. One patient with a T1b tumour was excluded from this analysis due to small sample size. c) Scatterplot showing correlation between percent genome altered and pre-treatment PSA values. Pearson (R) and Spearman (p) correlation values are displayed, with their respective P values. d) Heatmap of the contingency table of cluster and T category for 284 samples. P value is from a chi-squared test. e) Heatmap of the contingency table of cluster and Gleason score for 284 samples. P value is from a chi-squared test. f) Box plot comparing age at treatment between clusters. Grey dots represent values for individual samples. P value shown is from a one-way ANOVA. g) Box plot comparing PSA among clusters. Grey dots represent values for individual samples. P value shown is from a one-way ANOVA.

FIG. 19 shows Power analysis of CNAs. A power test for two proportions of different sample sizes was used to calculate the power we have for CNAs for various Gleason score groups. Heatmaps a-d show effect sizes of 0.2, 0.4, 0.6, and 0.8, respectively. The coloured dots represent the sample sizes we have in the various Gleason score group comparisons, with group 1 on the x-axis and group 2 on the y-axis. Background is symmetric and represents power, with values indicated by the colour key.

FIG. 20 shows Power analyses of SNVs and ncSNVs. a) Statistical power for detection of recurrent somatic coding SNVs. The curves represent the number of tumour/normal pairs needed to detect about 90% of significantly mutated genes with about 90% power, for various mutation rates in samples (0.5%, 1%, 2%, 3%, 5% and 10%) as a function of the background somatic mutation frequency per Mbp. The blue region represents the median background mutation frequency range, from 2.5th to 97.5th quantile, of CDS' in prostate cancer. b) Statistical power for detection of recurrent somatic non-coding SNVs. The curves represent the power at various samples sizes (0-1000) for various mutation rates (p1=0.5%, 1%, 2%, 3%, 5% and 10%) as compared to the background mutation rate at the median: 2.59×10⁻⁴. Vertical dashed lines at the sample sizes of 57 and 200 represent the WGS sample sizes presented in the Baca et al. and the current study, respectively. c) Statistical power for detection of recurrent somatic GRs. Power analysis of CTXs was calculated by dividing the genome into 3,113 bins of 1 Mbp each. A bin is considered as changed if we observe a CTX breakpoint within it (i.e. a bin is set to 1 if a breakpoint is observed, 0 otherwise). The background mutation rate is calculated as the proportion of recurrently mutated bins, where recurrent implies 3 or more samples give evidence to that mutation. Visualized as power by sample size (5 to 1000) for various frequencies of recurrence (2%, 3%, 5%, and 10%), with vertical lines at sample sizes of Baca et al. and the current study (57 and 200 respectively).

FIG. 21 shows Comparison of somatic coding SNV load across Gleason scores. Boxplot comparing the number of somatic coding SNVs by Gleason Score for 477 patients (p=2.47×10⁻³; one-way ANOVA). Grey dots represent values for individual samples.

FIG. 22 shows Association between ncSNV frequency and replication time. Replication time was plotted for each of the most recurrent ncSNVs. Replication timing data were available for 68/70 recurrent ncSNVs. Spearman (p) correlation and its P value are displayed.

FIG. 23 shows Transcription factor binding sites 1 Kbp flank. TFBS bias in aberrations. To determine if ncSNVs were biased towards specific transcription-factor binding sites (TFBSs), we tested if experimentally-derived TFBS locations from ENCODE were enriched for aberrations of different types using the binomial test. Heatmap of 58 TFBS cell lines for each sample coloured by the data type or combination of data types (SNV, CNV, and CTX flanked by 1 kbp) if it is aberrant in more than expected by chance (Binomial test with FDR adjusted P values). The samples are ordered by the number of significantly aberrant TFBSs (top barplot), the TFBS cell lines are ordered by fraction of samples with significantly mutated TFBSs by cell line (right barplot), covariates of Gleason score, pre-treatment PSA, T category, and patient age are displayed at the bottom.

FIG. 24 shows Trinucleotide mutation profiles. a) Percentage of SNV mutations attributed to a specific trinucleotide mutation category, for each normalized NMF-derived signature b) The contribution of each signature to the somatic SNV profile of each patient.

FIG. 25 shows Cross-individual contamination level. Heatmap showing the cross-individual contamination levels (in percentage) of the 130 CPC-GENE tumour-normal pairs were predicted by ContEst (v.1.0.24530). Each sample was sequenced in 2-18 flow-cell lanes. White indicates that the degree of cross-individual contamination in each lane was less than 2.5% (grey indicates no data). Sample-level contamination (normal_total, tumour_total) was also determined. Two normal samples had 2.5-5.0% of cross-individual contamination while no tumour samples had more than 2.5% of cross-individual contamination.

FIG. 26 shows suitable configured computer device, and associated communications networks, devices, software and firmware to provide a platform for enabling one or more embodiments as described herein.

### DETAILED DESCRIPTION

Herein we report the largest prostate cancer whole-genome sequencing cohort to date: 200 non-indolent localized specimens. We provide saturating discovery of recurrent driver single nucleotide variants (SNVs), copy number aberrations (CNAs) and genomic rearrangements (GRs) in this ethnic and clinical group, and associate these with epigenomic profiles. Many well-characterized recurrent molecular aberrations are confirmed, and novel prognostic translocations, inversions and epigenetic events are identified.

Specifically, we analyzed 200 whole-genome sequences and 477 whole-exome sequences from localized, non-indolent prostate tumours. These were supplemented with RNA and methylation analyses in a subset of cases. All tumours had similar pre-clinical risk profiles, reflecting the most common disease state on initial clinical presentation. These tumours have a paucity of clinically-actionable SNVs, unlike those seen in metastatic disease. Rather, a significant proportion of tumours harbour recurrent non-coding aberrations, large-scale genomic rearrangements, and a novel mode whereby an inversion represses transcription within its boundaries. Local hypermutation events (kataegis and chromothripsis) were frequent, and correlated with specific genomic profiles. Numerous molecular aberrations were prognostic for disease recurrence, including several DNA methylation events. These outperformed well-described prognostic biomarkers like MYC amplification, NKX3-1 and PTEN deletion, and percentage genome alteration. Our data suggest that novel therapeutic approaches should focus on recurrent targets in localized prostate cancer to improve cures in aggressive localized disease.

From these analyses a multimodal signature for prostate cancer was developed.

In a general aspect, there is provided a method of prognosing and/or predicting disease progression in subject with prostate cancer. The method comprises use of at least 2 patient biomarkers determined or measured from genetic material of cancer cells and comparing them to corresponding reference or control measures of the same biomarkers. The biomarkers are selected from T category, and aberrations in ACTL6B, TCERGL1, chr7:61 Mbp, ATM and MYC. Statistically significant aberrations of the subject biomarkers when compared to the reference biomarkers would be indicative of a worse outcome.

The methods described herein are useful for prognosing the outcome of a subject that has, or has had, a cancer associated with the prostate. The cancer may be prostate cancer or a cancer that has metastasized from a cancer of the prostate.

In an aspect, there is provided a method of prognosing and/or predicting disease progression in subject with prostate cancer, the method comprising: a) providing a sample containing genetic material from cancer cells; b)determining or measuring at least 2 patient biomarkers regarding the prostate cancer tumor selected from the group consisting of: T category, ACTL6B methylation, TCERGL1 methylation, chr7:61 Mbp inter-chromosomal translocation, ATM single nucleotide variants and MYC copy number aberrations; c) comparing said patient biomarkers to corresponding reference or control biomarkers; and d) determining the likelihood of disease progression; wherein a likelihood of disease progression is higher with each of ACTL6B hyper-methylation, TCERGL1 hypo-methylation, higher T category, and higher incidences of chr7:61 Mbp inter-chromosomal translocation, ATM single nucleotide variants and MYC copy number aberrations, when the difference is statistically significant on comparison with the reference or control biomarkers.

The term “subject” as used herein refers to any member of the animal kingdom, preferably a human being and most preferably a human being that has, has had, or is suspected of having prostate cancer.

The term “sample” as used herein refers to any fluid (e.g. blood, urine, semen), cell, tumor or tissue sample from a subject which can be assayed for the biomarkers described herein.

The term “genetic material” used herein refers to materials found/originate in the nucleus, mitochondria and cytoplasm, which play a fundamental role in determining the structure and nature of cell substances, and capable of self-propagating and variation. In the context of the present methods, the genetic material is any material from which one can measure the biomakers described herein. The genetic material is preferably DNA.

A “genetic aberration” is any change in genetic material that is unusual or uncommon when compared to wild-type or control genetic material. Genetic aberrations include deletions, substitutions, insertions, SNVs, translocations, hyper or hypo-methylation, copy number abberations and any other genetic mutations.

The term “prognosis” as used herein refers to the prediction of a clinical outcome associated with a disease subtype which is reflected by a reference profile such as a biomarker reference profile. The prognosis provides an indication of disease progression and includes an indication of likelihood of death due to cancer. The prognosis may be a prediction of metastasis, or alternatively disease recurrence. In one embodiment the clinical outcome class includes a better survival group and a worse survival group. The term “prognosing or classifying” as used herein means predicting or identifying the clinical outcome of a subject according to the subject's similarity to a reference profile or biomarker associated with the prognosis. For example, prognosing or classifying comprises a method or process of determining whether an individual has a better or worse survival outcome, or grouping individuals into a better survival group or a worse survival group, or predicting whether or not an individual will respond to therapy.

In various embodiments, the at least 2 patient biomarkers, are at least 3, 4, 5 or 6 patient biomarkers.

In some embodiments, the prostate cancer is localized prostate cancer, preferably non-indolent localized prostate cancer.

In some embodiments, the method further comprises building a patient biomarker profile from the determined or measured patient biomarkers.

The term “biomarker profile” as used herein refers to a dataset representing the state or expression level(s) of one or more biomarkers. A biomarker profile may represent one subject, or alternatively a consolidated dataset of a cohort of subjects, for example to establish a reference biomarker profile as a control.

As used herein, the term “control” refers to a specific value or dataset that can be used to prognose or classify the value e.g the measured biomarker or reference biomarker profile obtained from the test sample associated with an outcome. In one embodiment, a dataset may be obtained from samples from a group of subjects known to have cancer having different tumor states and/or healthy individuals. The state or expression data of the biomarkers in the dataset can be used to create a control value that is used in testing samples from new patients. In some embodiments, a cohort of subjects is used to obtain a control dataset. A control cohort patients may be a group of individuals with or without cancer.

In some embodiments, the prediction of disease progression is following at least one of surgery, endocrine therapy, chemotherapy, radiotherapy, hormone therapy, gene therapy, thermal therapy, and ultrasound therapy.

In some embodiments, the method further comprises classifying the patient into a high risk group if the likelihood of disease progression is relatively high or a low risk group if the likelihood of disease progression is relatively low.

As used herein, “overall survival” refers to the percentage of or length of time that people in a study or treatment group are still alive following from either the date of diagnosis or the start of treatment for a disease, such as cancer. In a clinical trial, measuring the overall survival is one way to see how well a new treatment works.

As used herein, “relapse-free survival” refers to, in the case of caner, the percentage of or length of time that people in a study or treatment group survive without any signs or symptoms of that cancer after primary treatment for that cancer. In a clinical trial, measuring the relapse-free survival is one way to see how well a new treatment works. It is defined as any disease recurrence or relapse (local, regional, or distant).

The term “good survival” or “better survival” as used herein refers to an increased chance of survival as compared to patients in the “poor survival” group. For example, the biomarkers of the application can prognose or classify patients into a “good survival group”. These patients are at a lower risk of death after surgery and can also be categorized into a “low-risk group”.

The term “poor survival” or “worse survival” as used herein refers to an increased risk of disease progression or death as compared to patients in the “good survival” group. For example, biomarkers or genes of the application can prognose or classify patients into a “poor survival group”. These patients are at greater risk of death or adverse reaction from disease or surgery, treatment for the disease or other causes, and can also be categorized into a “high-risk group”.

A person skilled in the art would understand how to implement differing cut-offs for good survival vs. worse survival, depending on the clinical outcome one is predicting and the biomarkers being assayed.

In some embodiments, the method further comprises treating the patient with more aggressive therapy if the patient is in the high risk group.

In some embodiments, the more aggressive therapy comprises adjuvant therapy, preferably hormone therapy, chemotherapy or radiotherapy.

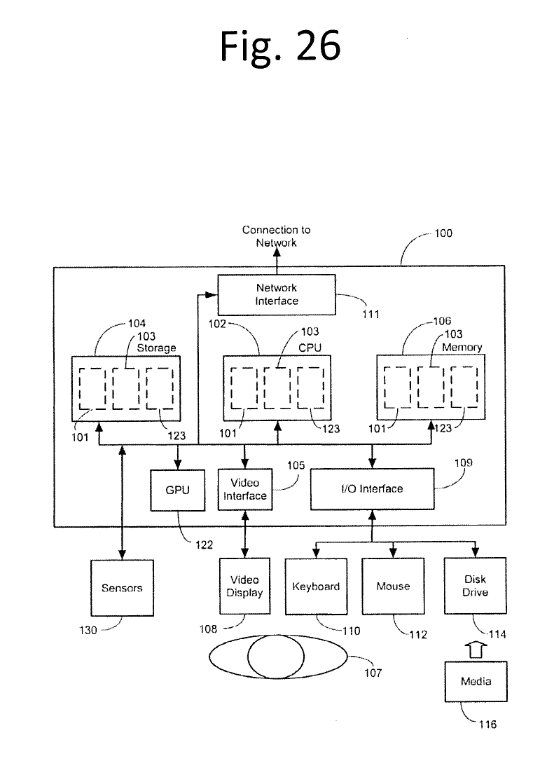

The present system and method may be practiced in various embodiments. A suitably configured computer device, and associated communications networks, devices, software and firmware may provide a platform for enabling one or more embodiments as described above. By way of example, FIG. 26 shows a generic computer device 100 that may include a central processing unit (“CPU”) 102 connected to a storage unit 104 and to a random access memory 106. The CPU 102 may process an operating system 101, application program 103, and data 123. The operating system 101, application program 103, and data 123 may be stored in storage unit 104 and loaded into memory 106, as may be required. Computer device 100 may further include a graphics processing unit (GPU) 122 which is operatively connected to CPU 102 and to memory 106 to offload intensive image processing calculations from CPU 102 and run these calculations in parallel with CPU 102. An operator 107 may interact with the computer device 100 using a video display 108 connected by a video interface 105, and various input/output devices such as a keyboard 115, mouse 112, and disk drive or solid state drive 114 connected by an I/O interface 109. In known manner, the mouse 112 may be configured to control movement of a cursor in the video display 108, and to operate various graphical user interface (GUI) controls appearing in the video display 108 with a mouse button. The disk drive or solid state drive 114 may be configured to accept computer readable media 116. The computer device 100 may form part of a network via a network interface 111, allowing the computer device 100 to communicate with other suitably configured data processing systems (not shown). One or more different types of sensors 135 may be used to receive input from various sources.

The present system and method may be practiced on virtually any manner of computer device including a desktop computer, laptop computer, tablet computer or wireless handheld. The present system and method may also be implemented as a computer-readable/useable medium that includes computer program code to enable one or more computer devices to implement each of the various process steps in a method in accordance with the present invention. In case of more than computer devices performing the entire operation, the computer devices are networked to distribute the various steps of the operation. It is understood that the terms computer-readable medium or computer useable medium comprises one or more of any type of physical embodiment of the program code. In particular, the computer-readable/useable medium can comprise program code embodied on one or more portable storage articles of manufacture (e.g. an optical disc, a magnetic disk, a tape, etc.), on one or more data storage portioned of a computing device, such as memory associated with a computer and/or a storage system.

In an aspect, there is provided a computer-implemented method of predicting disease progression in patient with prostate cancer, the method comprising: a) receiving, at at least one processor, data reflecting at least 2 patient biomarkers regarding the prostate cancer tumor selected from the group consisting of: T category, ACTL6B methylation, TCERGL1 methylation, chr7:61 Mbp inter-chromosomal translocation, ATM single nucleotide variants and MYC copy number aberrations; b) constructing, at the at least one processor, an expression profile corresponding to the expression levels; c) comparing, at the at least one processor, said patient biomarkers to corresponding reference or control biomarkers; d) determining, at the at least one processor, the likelihood of disease progression; wherein a likelihood of disease progression is higher with each of ACTL6B hyper-methylation, TCERGL1 hypo-methylation, higher T category, and higher incidences of chr7:61 Mbp inter-chromosomal translocation, ATM single nucleotide variants and MYC copy number aberrations, when the difference is statistically significant on comparison with the reference or control biomarkers.

In an aspect, there is provided a computer program product for use in conjunction with a general-purpose computer having a processor and a memory connected to the processor, the computer program product comprising a computer readable storage medium having a computer mechanism encoded thereon, wherein the computer program mechanism may be loaded into the memory of the computer and cause the computer to carry out the method described herein.

In an aspect, there is provided a computer readable medium having stored thereon a data structure for storing the computer program product described herein.

In an aspect, there is provided a device for predicting disease progression in patient with prostate cancer, the device comprising: at least one processor; and electronic memory in communication with the at one processor, the electronic memory storing processor-executable code that, when executed at the at least one processor, causes the at least one processor to: a) receive data reflecting at least 2 patient biomarkers regarding the prostate cancer tumor selected from the group consisting of: T category, ACTL6B methylation, TCERGL1 methylation, chr7:61 Mbp inter-chromosomal translocation, ATM single nucleotide variants and MYC copy number aberrations; b) compare said patient biomarkers to corresponding reference or control biomarkers; and c) determining, at the at least one processor, the likelihood of disease progression; wherein a likelihood of disease progression is higher with each of ACTL6B hyper-methylation, TCERGL1 hypo-methylation, higher T category, and higher incidences of chr7:61 Mbp inter-chromosomal translocation, ATM single nucleotide variants and MYC copy number aberrations, when the difference is statistically significant on comparison with the reference or control biomarkers.

As used herein, “processor” may be any type of processor, such as, for example, any type of general-purpose microprocessor or microcontroller (e.g., an Intel™ x86, PowerPC™, ARM™ processor, or the like), a digital signal processing (DSP) processor, an integrated circuit, a field programmable gate array (FPGA), or any combination thereof.

As used herein “memory” may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), or the like. Portions of memory 102 may be organized using a conventional filesystem, controlled and administered by an operating system governing overall operation of a device.

As used herein, “computer readable storage medium” (also referred to as a machine-readable medium, a processor-readable medium, or a computer usable medium having a computer-readable program code embodied therein) is a medium capable of storing data in a format readable by a computer or machine. The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The computer readable storage medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the computer readable storage medium. The instructions stored on the computer readable storage medium can be executed by a processor or other suitable processing device, and can interface with circuitry to perform the described tasks.

As used herein, “data structure” a particular way of organizing data in a computer so that it can be used efficiently. Data structures can implement one or more particular abstract data types (ADT), which specify the operations that can be performed on a data structure and the computational complexity of those operations. In comparison, a data structure is a concrete implementation of the specification provided by an ADT.

The above listed aspects and/or embodiments may be combined in various combinations as appreciated by a person of skill in the art. The advantages of the present disclosure are further illustrated by the following examples. The examples and their particular details set forth herein are presented for illustration only and should not be construed as a limitation on the claims of the present invention.

### EXAMPLES

### Saturating Genomic Interrogations of Localized Prostate Cancer

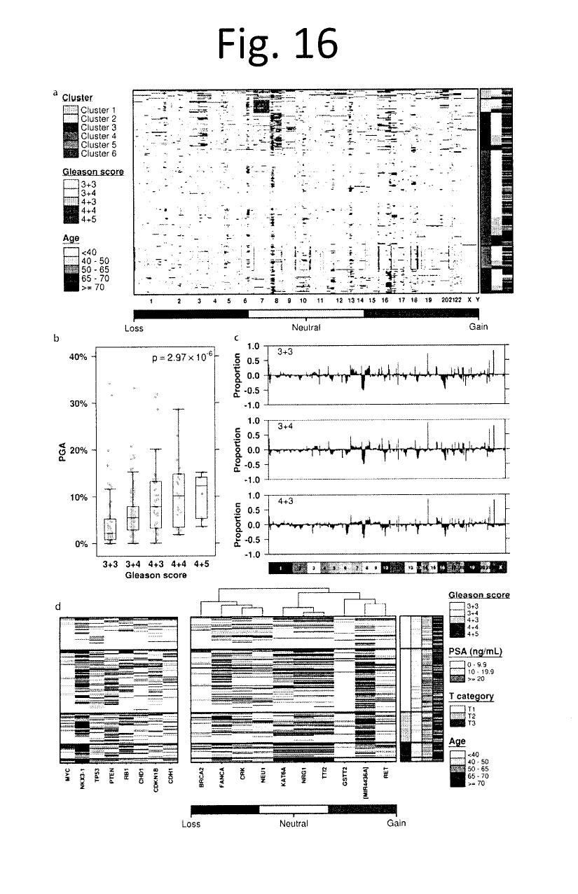

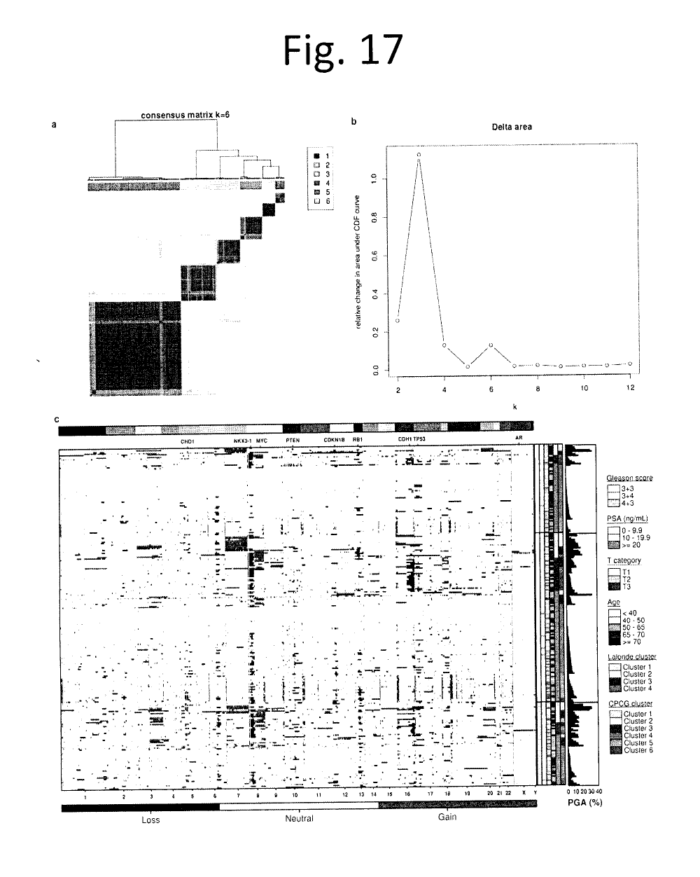

To address the genetic heterogeneity of non-indolent localized disease, we first comprehensively profiled CNAs in 284 localized prostate adenocarcinomas (FIG. 16 and data not shown). The profiles recapitulated those reported in prior studies, including recurrent allelic gains of MYC and allelic deletions of PTEN, TP53 and NKX3-1 (FIGS. 17-19 and data not shown). Even in this clinically homogeneous population, we observed large inter-tumoural heterogeneity in the percentage of the genome with a CNA (PGA), ranging from 0-39.2%⁴.

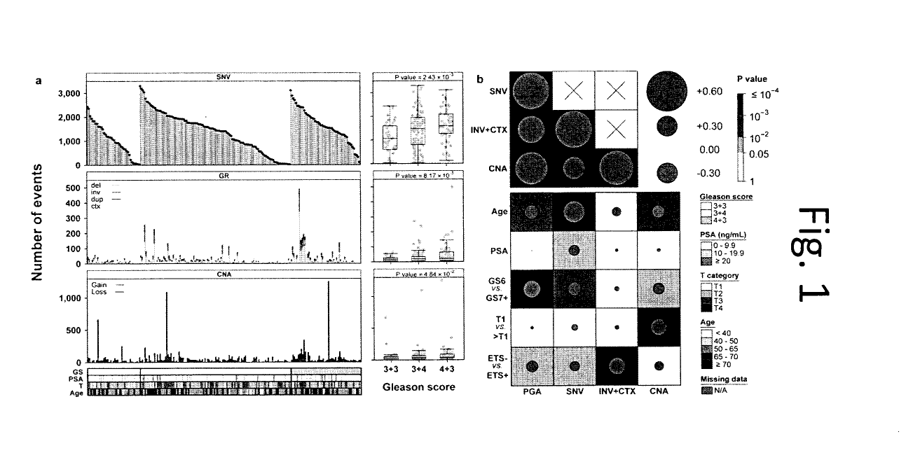

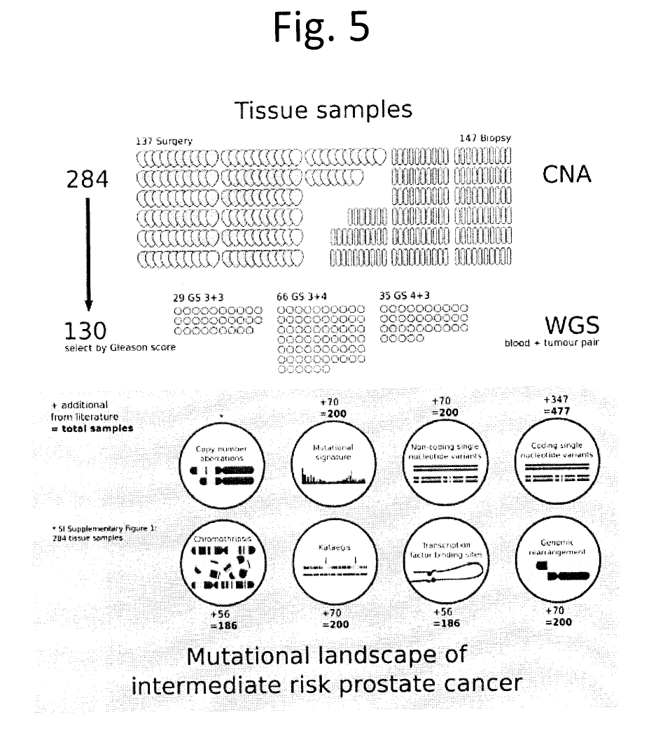

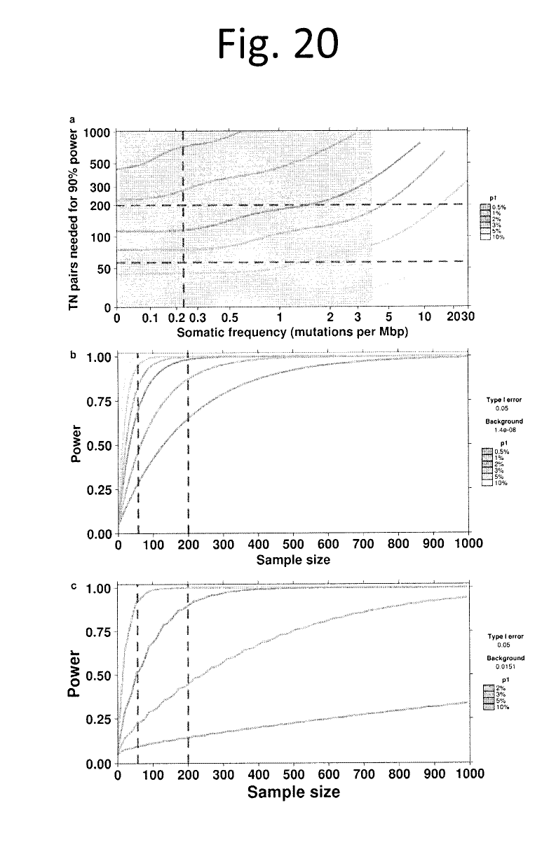

We next performed high-depth whole-genome sequencing (WGS) of 130 of these tumours (and matched bloods), focusing on localized tumours amenable to surgery (i.e. Gleason Score (GS) of 3+3, 3+4 or 4+3). These were supplemented by 70 tumour/normal pairs with publicly-available read-level WGS data⁹⁻¹² and 277 read-level exome sequences^{9,10,12,13}, all with similar GSs. WGS data covered 84.2±2.5% (mean±standard-deviation) of the non-repetitive genome to at least 17x tumour and 10x normal (67.1-85.7%), allowing robust analysis of the entire genome. All samples were aligned and profiled for SNVs and GRs, using well characterized and validated pipelines (¹⁴ and Lee et al. in review; FIG. 1a and data not shown). Overall, this yielded 477 prostate tumours with analysis of somatic coding SNVs (data not shown), of which 200 had whole-genome sequencing (FIG. 5, FIG. 1a). These data give 62.9-99.9% power to detect recurrent coding and non-coding SNVs at the 0.5-10% levels¹⁵ (FIGS. 20a,b). Similarly we had >99.9% power to detect Genomic Rearrangements (GRs) present at 10% recurrence and 44.7% power for 3% recurrence (FIG. 20c). To supplement these genomic metrics, we performed RNA abundance profiling on 73 tumours and methylation profiling on 104, and generated methylation subtypes through unsupervised machine-learning (data not shown).

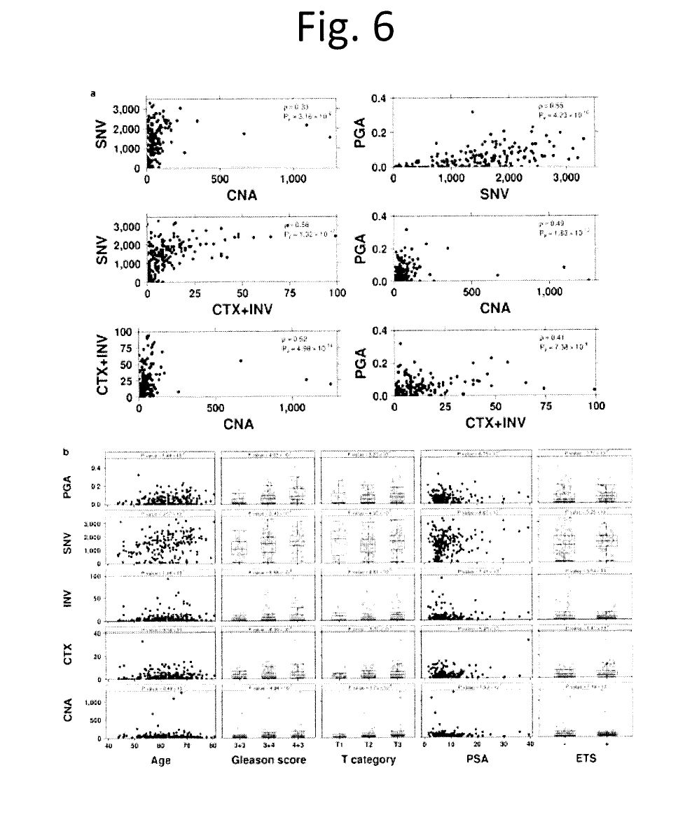

We observed a low overall SNV burden, with a median 0.53 (0.05-6.92) somatic SNVs/Mbp across all tumours (FIG. 1a). SNV burden was significantly elevated in tumours containing Gleason pattern 4, with a median of 1,063 in GS 3+3, 1,482 in 3+4 and 1,585 in 4+3 tumours (p=1.05×10⁻³; t-test). The number of GRs was highly variable across tumours, with a median of 19 (0-499) and those with any GS 4 component (i.e. 3+4 or 4+3) showed elevated rates (median 17 GRs in GS 3+3 vs. 22 in GS 3+4 and 4+3; p=5.11×10⁻⁴; t-test). The number of inversions and translocations were correlated with SNV burden (FIG. 1b, FIG. 6a; p=0.55, p=7.93×10⁻¹⁷). Several other associations between mutation burden and clinical covariates like PSA, tumour size and ETS fusions were uncovered (FIG. 6).

### Somatic SNV Profiles

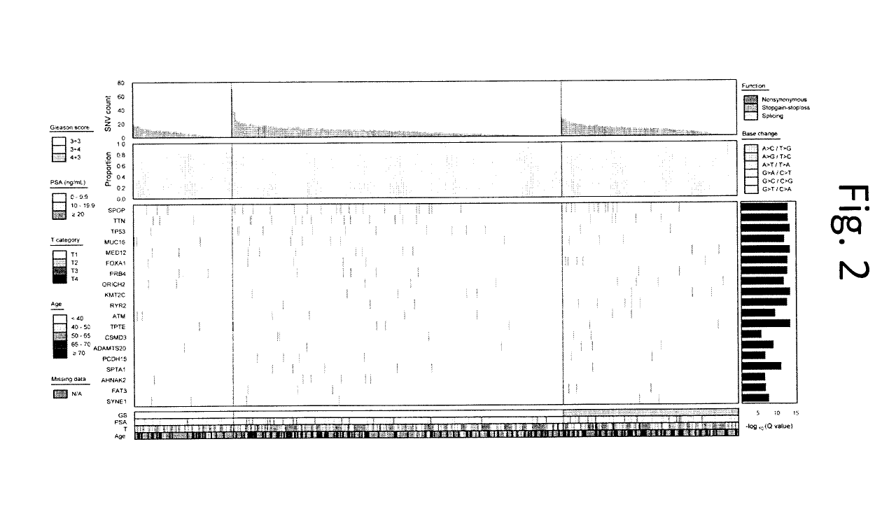

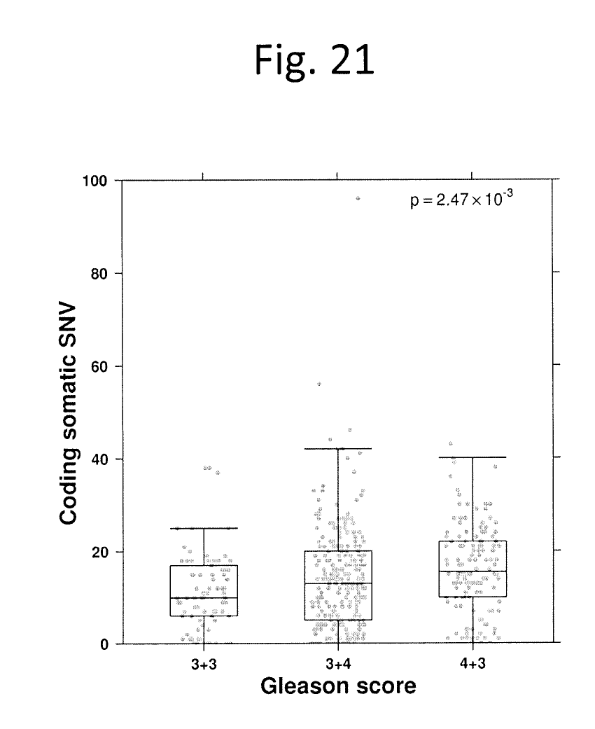

Individual tumours harboured 0-98 exomic SNVs (FIG. 2). The median number of non-synonymous mutations increased with GS (3+3-7; 3+4-9; 4+3-10; p=0.001, one-way ANOVA; FIG. 21 and data not shown). Only six genes were mutated at a rate of >2%: SPOP (8.0%; 38/477), TTN (4.4%, 21/477), TP53 (3.4%; 16/477), MUC16 (2.5%; 12/477), MED12 (2.3%; 11/477) and FOXA1 (2.3%; 11/477). The AR gene was altered by non-synonymous SNVs in only 2/477 tumours (a GS 3+3 and a GS 3+4), while allelic deletions in AR were observed in 4/284 tumours and amplification in 1/284 tumours. Interestingly, 8 tumours (1.75%) harboured mutations in the critical DNA damage checkpoint activator, ATM. Mutations in several genes were associated with GS, most prominently FAT1 (0/78 GS 3+3; 0/261 GS 3+4; 5/133 GS 4+3; p=0.0048; Fisher's Exact Test). Similarly mutations in multiple genes were associated with increased genomic instability as measured by PGA, including MYO15A (2.7% in wildtype vs. 6.3% in mutated; FDR p=1.01×10⁻¹¹). Based on the median background rate of 2.59×10⁴ for coding regions, we estimate that there remain no genes to be discovered that are mutated at ≥1% rate, but ˜5 undiscovered genes mutated at the 0.5% level. The low frequency of these mutations juxtaposed with the high rate of CNAs confirms the C-class character of prostate cancers.

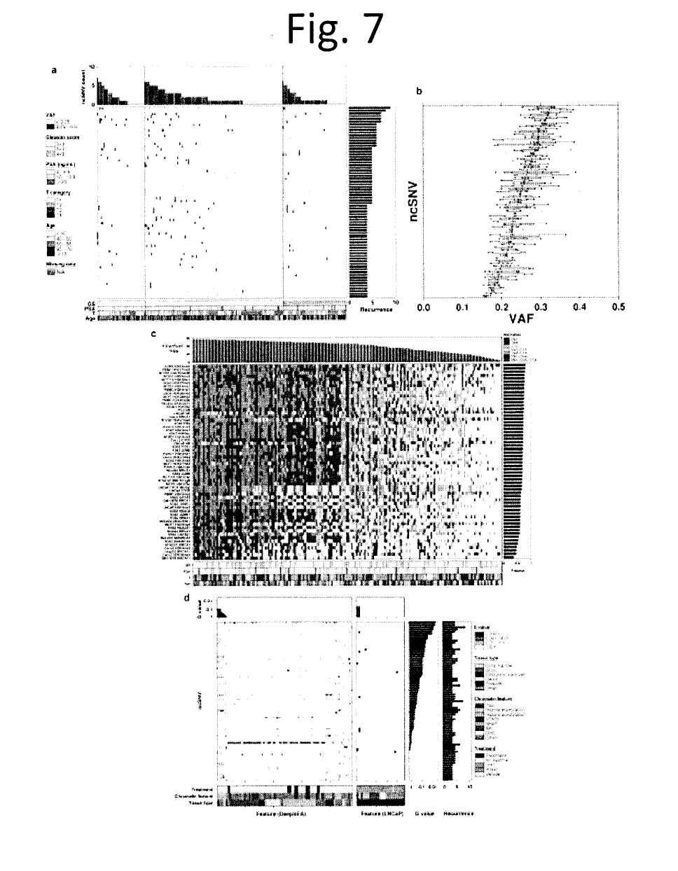

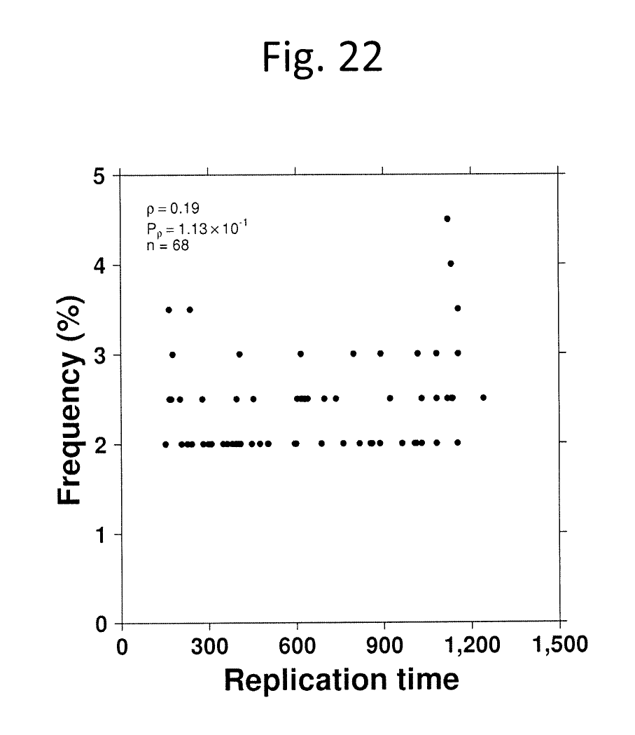

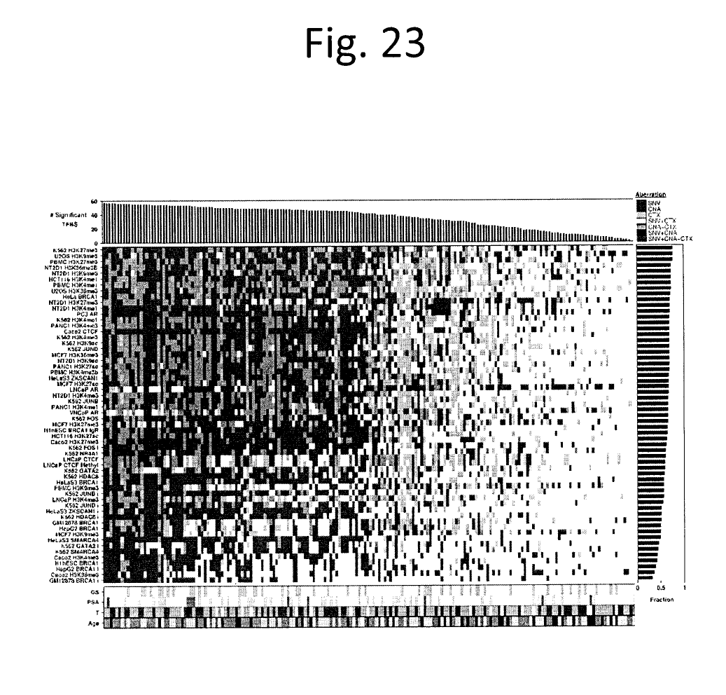

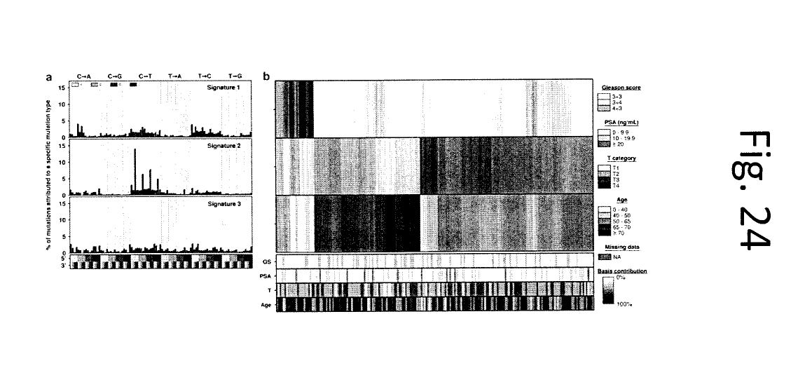

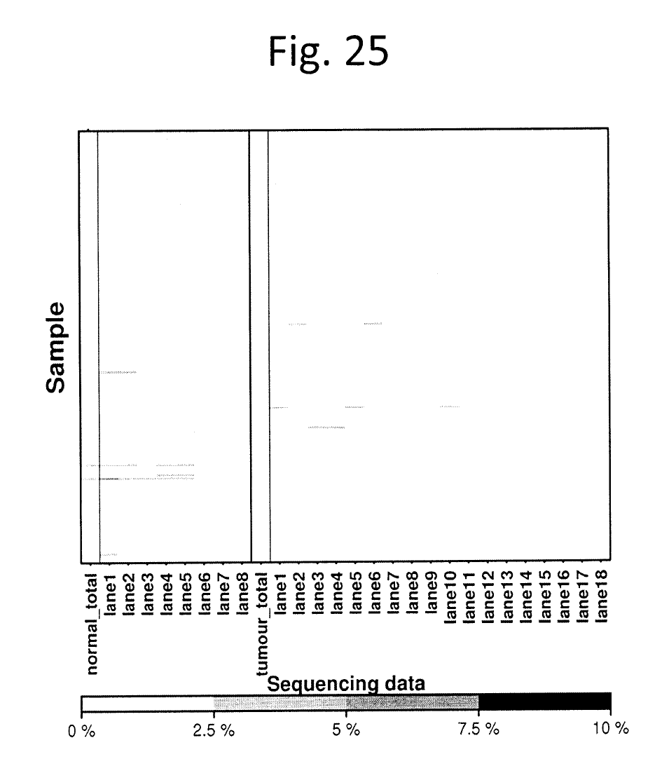

We next explored the non-coding regions of the genome in the 200 tumours with WGS. Multiple recurrent ncSNVs (i.e. identical genomic position) were detected: 7 ncSNVs were observed in at least 7/200 patients, while another 63 were mutated in 4-6 patients. These SNVs are thus altered at a similar ˜2-4% mutation rate as TP53, MED12 and FOXA1 (FIG. 7a). Most tumours harboured at least one recurrent ncSNV (2% recurrence or higher; median: 1/tumour). There was a strong bias in trinucleotide context towards TCT/AGA trinucleotides (27/70 SNPs). Several ncSNVs showed trend associations with GS, PGA and ETS fusions, highlighting a potential role in driving mutational phenotypes, and the need for larger cohorts to uncover these effects. Recurrent ncSNVs were not associated with replication time (FIG. 22), and encompassed a broad range of variant allele frequencies from clonal to small subclones (FIG. 7b). Recurrent ncSNVs did not generally localize to specific transcription-factor binding-sites, although GRs and CNAs did (FIG. 7c, FIG. 23). We therefore considered the potential impact of SNVs on chromatin structure, both across a wide range of marks from multiple cell-types using DeepSEA¹⁷ and in a panel of 14 marks characterized in the LNCaP prostate cancer-derived cell line (FIG. 7d and data not shown). Overall 6/70 recurrent ncSNVs showed evidence of perturbing chromatin structure at q<0.01, while no individual chromatin feature was significantly enriched across ncSNVs.

Next we sought to assess mutational signatures by considering the trinucleotide profiles of somatic SNVs using non-negative matrix factorization¹⁸. Three distinct trinucleotide signatures were identified from WGS data (FIG. 24a and data not shown). Signature 2 reflects the deamination profile previously reported as a hallmark of sequencing false positives^{14,18}. Increased expression of signature 2 showed a marginal positive association with T3 (β=0.398; q=0.044; glm) and a negative association with age (β=0.015; q=0.022; glm); signature 3 showed a weak positive association with age (β=0.014; q=0.049; glm). By contrast, Signatures 1 is characterized by a relatively uniform mutational profile and was not associated with age, Gleason score, PSA, or T category (data not shown). These signatures occur in individual patients at different frequencies (FIG. 24b; data not shown). Exposures of the signatures were Pearson correlated with collapsed CNA genes and GR (INV+CTX) genes that were recurrent in at least 5% of patients. We also observed CNA genes (at the 5% FDR corrected level) for each signature (data not shown). There were no significant correlations between the exposures of signatures and GRs.

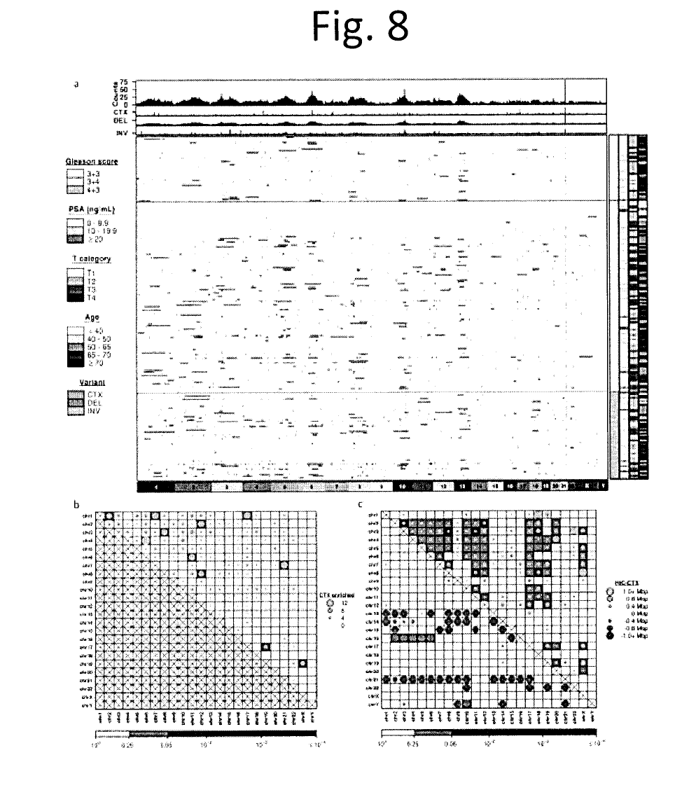

GRs have been poorly-studied in localized prostate cancer; however they may provide evidence for DNA double-stranded break events during progression. As expected, the TMPRSS2:ERG (T2E) fusion on chromosome 21 was the most recurrent GR, observed in 38% of tumours (76/200; FIG. 8a). Other frequent alterations include translocation of MMS22L (chr6q16.1) and ARHGAP10 (chr4q31.23) in 12/200 tumours and translocation of chr17p11.1 and chr1q21.2 in 7/200 tumours. These recurrent alterations were reflected by several chromosome-pairs being involved in more inter-chromosomal GRs than expected (8b), including some without prominent focal GR-peaks (e.g. chr4-chr6; expected 2 CTXs; observed 14 CTXs; q<0.001, permutation test). Anticipating that these effects might be induced by inter-chromosomal proximity^{19,20}, we compared pair-wise GR enrichment to Hi-C data measuring inter-chromosomal links in the RWPE1 prostate cancer cell-line²¹. Translocations between a few chromosome pairs co-localized with Hi-C links, but surprisingly many more were farther from Hi-C links than expected by chance (8c).

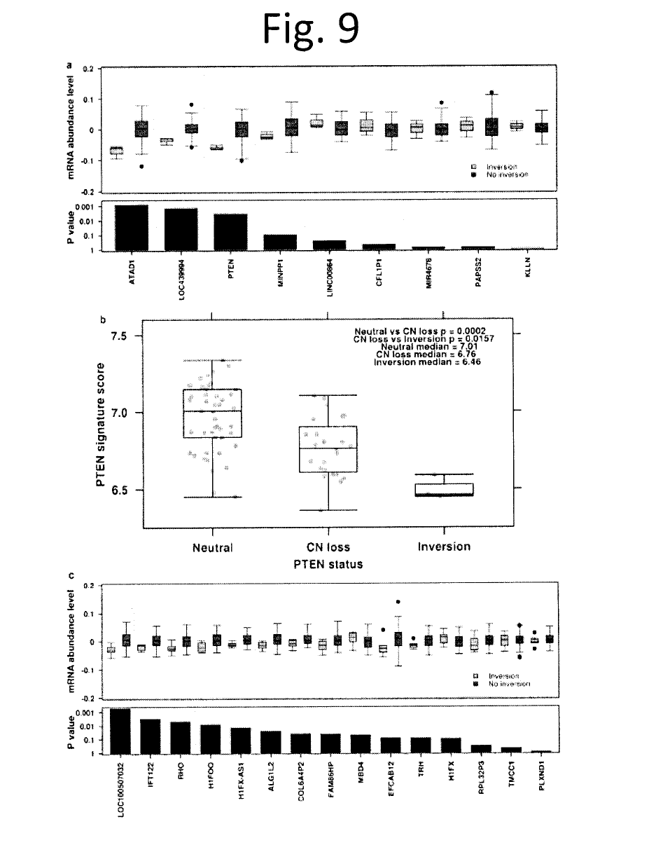

To further understand regional GR effects, we then divided the genome into 1 Mbp bins and considered the frequency of GRs in each (FIG. 8a and data not shown). Six bins had elevated rates of inversions: chr3:125-126 Mbp and chr3:129-130 Mbp contained inversions in 6% of patients (12/200), while chr10:89-90 Mbp contained inversions in 5.5% (11/200) and chr3:195-196 Mbp, chr21:39-40 Mbp and chr21:42-43 Mbp all contained inversions in 5% (10/200) of patients. A recurrent inversion on chr10:89-90 Mbp in 11/200 of patients, and was associated with a significant decrease in the mRNA abundance of three genes within it: LOC439994, ATAD1 and PTEN (FIG. 9a), suggesting a novel mode of PTEN repression. Patients with this inversion showed lower PTEN pathway activity than those with deletions of the gene (FIG. 9b). This mode may be more general than just the PTEN locus: inversions in the chr3:129-130 Mbp also significantly dysregulate mRNA abundance, with 8/15 genes repressed in tumours harbouring the inversion (p<0.05; limma; FIG. 9c).

### Localized Somatic Hyper-Mutation: Chromothripsis and Kataegis

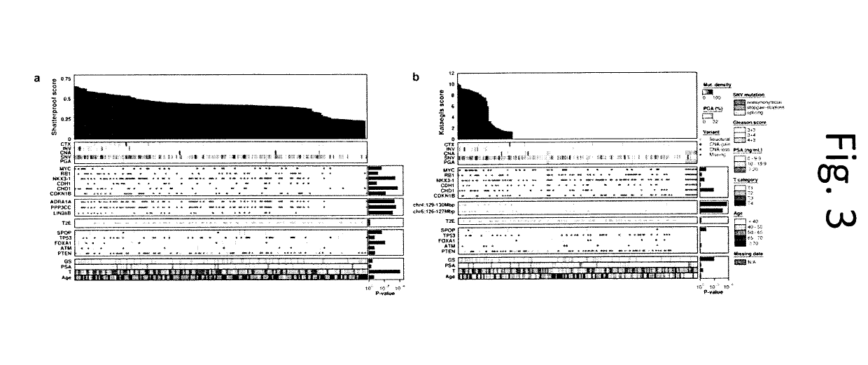

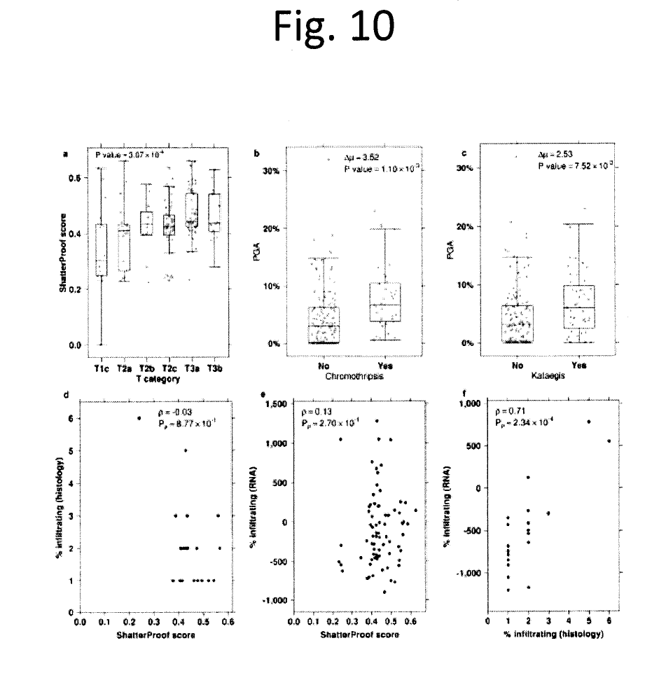

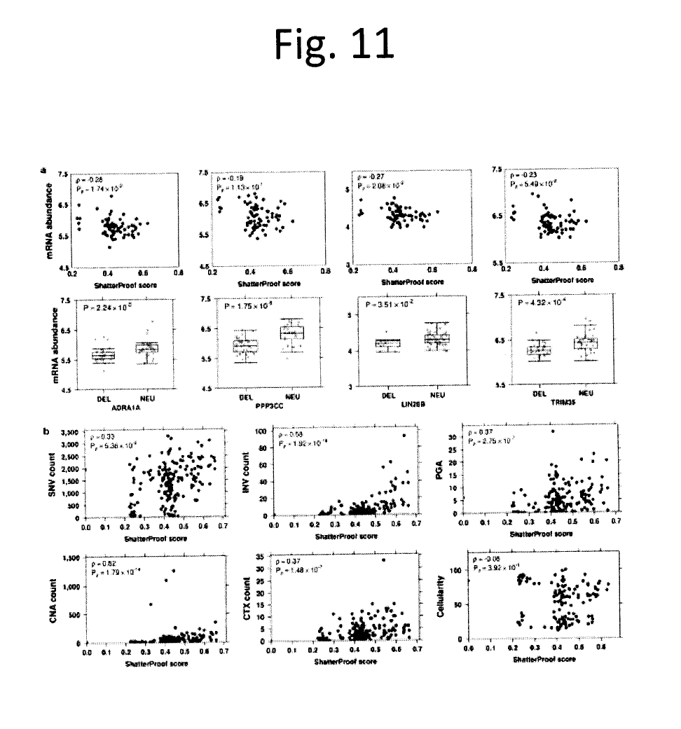

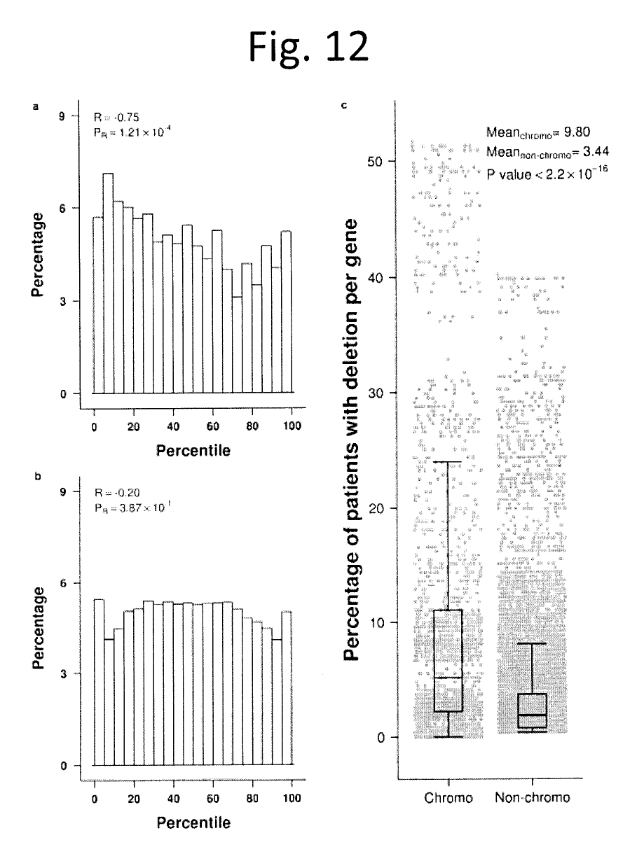

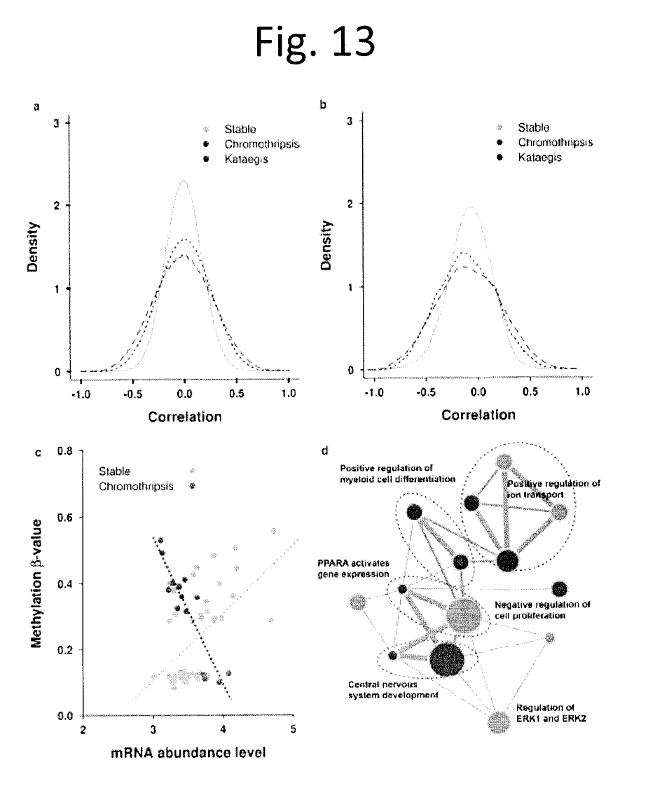

While some tumours are initiated or driven by recurrent point mutations in specific genes, others could be driven by focal genomic instability either at the level of DNA double-stranded breaks (i.e. chromothripsis²²) or DNA single-stranded breaks (i.e. kataegis¹⁸). Using ShatterProof²³ we detected chromothriptic characteristics in 20% (38/186) of tumours with CNA data (FIG. 3a and data not shown). These tumours were larger (Kendall's τ=0.23, p=3.07×10⁻⁴; FIG. 10a), but not associated with other clinical variables like age (p=0.24) or Gleason Score (p=0.35). The presence of chromothripsis was associated with both point mutations in FOXA1 (p=0.008) and CNAs in NKX3-1 (p=3.5×10⁻⁴), CHD1 (p=1.7×10⁻⁴) and CDKN1B (p=3.5×10⁻⁴) using Wilcoxon rank-sum test. Chromothriptic tumours were also significantly enriched for deletion of a locus on chr8 q36.32-p11.21 containing ADRA1A, PPP3CC and several genes other whose mRNA abundance was correlated with increased correlation to ShatterProof scores (FIG. 11a). The overall CNA burden was very modestly increased in chromothriptic tumours, as were essentially all mutation types, but not tumour cellularity (FIGS. 10b & 11b). Genes within chromothriptic regions largely showed reduced mRNA abundance but not methylation, and were dramatically enriched for genes deleted in other tumours suggesting that chromothripsis tends to inactivate tumour-suppressors (FIG. 12). Both promoter-region and global mRNA-methylation correlations were perturbed in chromothriptic tumours, and perturbed genes were enriched (FDR<5%) in pathways associated with development (FIG. 13 and data not shown). The mRNA abundances of 57 genes were strongly correlated with chromothripsis (|R|≥0.35; data not shown). Several immune genes were negatively correlated with chromothripsis, including the proto-oncogene DBL (also called MCF2, p=−0.43; p=2.0×10⁻⁴) and CD36 (p=−0.38; p=7.0×10⁻⁴), suggesting a possible role of immune dysregulation in chromothripsis, although few infiltrating immune cells were identified in primary tumours and their presence was not correlated to chromothriptic events either by histology or RNA signatures (FIG. 10d-f).

To quantify the presence of kataegis, we developed a sliding-window approach using the binomial test, a test for base change enrichment and an assessment of the expected proportion of variants within a given window. We detected kataegis in 46/200 samples (23%; FIG. 3b and data not shown). Kataegic tumours were significantly enriched for CHD1 deletion (15/45=33% with kataegis vs. 16/141=11.3% without kataegis; p=0.001; prop-test) Additionally, kataegis was also preferentially found in tumours with SNVs in SPOP (p=0.05; prop-test) or genomic rearrangements in regions on 4q (129-130 Mbp; FDR q=0.002; prop-test) or 6q (126-127 Mbp; FDR q=0.006; prop-test). Further, tumours with kataegic events showed significantly elevated genomic instability (FIG. 10c; p=7.52×10⁻³; t-test). Kataegis was associated with a clinically distinct subset of tumours, with elevated Gleason grade (13% of GS 3+3 samples having kataegic events vs. 19% of GS 3+4 and 39% of GS 4+3; Kendall's τ=0.21; FDR q=0.004).

### Recurrent Genomic and Epigenomic Aberrations Predict Patient Outcome

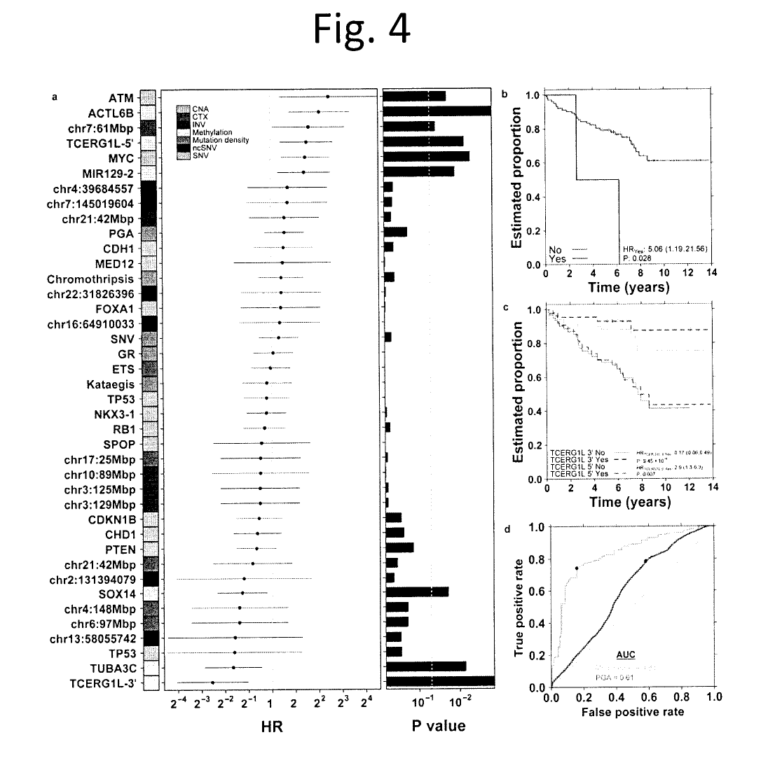

To better characterize the suite of recurrent events in localized prostate cancer, we next evaluated the association of each of these with patient survival. Of our patients with whole-genome sequencing, 130/200 had available data on disease relapse, as measured by biochemical recurrence (BCR, rise of PSA levels following primary therapy, see methods), with a median 7.96-year follow-up. We systematically evaluated the clinical relevance of 40 recurrent genomic alterations in localized prostate cancer: 3 measures of mutation density, kataegis, chromothripsis, 5 recurrent coding SNVs, 6 recurrent non-coding SNVs, 6 methylation events, 6 recurrent translocations, 4 recurrent inversions, and 8 CNAs. For each of these we employed univariate CoxPH modeling (FIG. 4a). Only one SNV was predictive of patient outcome: all patients with point mutations in ATM suffered relapse (FIG. 4b). A recurrent inter-chromosomal translocation breakpoint at the chromosome 7 centromere (chr7:61-62 Mbp) and the well-known amplification of MYC oncogene were also prognostic events²⁴. By contrast in this cohort, no measures of mutation intensity (i.e. PGA or the number of GRs or SNVs) nor of mutation density (i.e. chromothripsis or kataegis) were associated with patient outcome, although PGA showed a strong trend effect.

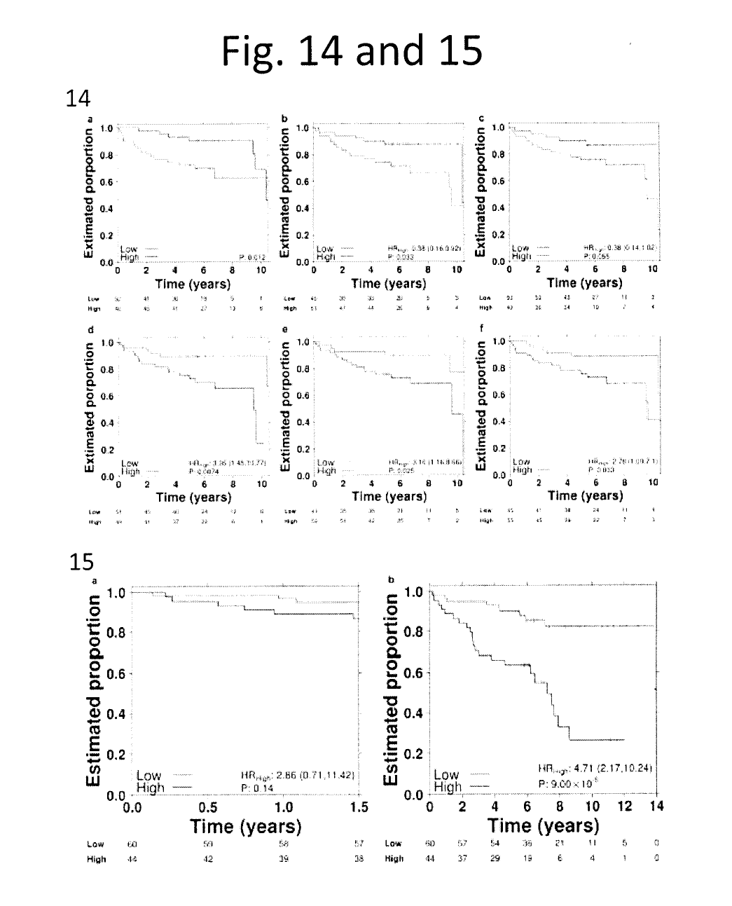

Remarkably, methylation status was tightly associated with patient outcome, much more so than any other genomic characteristic: of the 9 events significantly (p<0.05; Wald test) associated with disease recurrence, 6 involved DNA methylation. For example, hyper-methylation of a probe 5′ of a transcriptional elongation regulator (TCERG1L) shows a strong association with poor outcome (HR=2.90; 95% CI: 1.30-6.30; p=0.007). Fascinatingly, another probe on the 3′ end of TCERG1L showed the inverse association, with hypo-methylation associated with good outcome (HR=0.17; 95% CI: 0.06-0.49; p=9.45×10⁻⁴; FIG. 4c). Of the six prognostic methylation events, five were validated in an independent cohort of 100 intermediate-risk patients (FIG. 14).

Finally, we evaluated whether these diverse events could be integrated into a multi-modal, DNA-based biomarker to predict the disease relapse. Such a biomarker would be of significant clinical value, as many of these patients are over-treated in current management. We applied multivariate CoxPH modeling using cross-validation to test the outcome of a multi-modal biomarker: T category, ACTL6B hyper-methylation, TCERGLI hypo-methylation, the chr7:61 Mbp CTX, ATM SNVs, and MYC CNA. This signature was highly discriminative of patients who would experience disease relapse, with an Area Under the ROC Curve of 0.83 (95% CI: 0.80-0.86, as compared to that of 0.61 for the validated PGA biomarker (FIG. 4d)^{6,25}, and with a concordance index of 0.79. This discriminative ability predicted robust differences in patient survival (HR=4.71; p=9.00×10⁻⁹; Wald test; FIG. 15).

### Analysis

Localized, non-indolent prostate cancer is the most common state at initial clinical presentation. We used whole-genome sequencing to identify a series of recurrent mutational events outside of the exome. Because of the paucity of driver and prognostic coding aberrations, consideration of the entire prostate cancer genome may be critical in biomarker studies to find driver aberrations missed in smaller studies^{4,10}. For example, we identify several inversions associated with mRNA abundance decreases, potentially representing a novel mode of tumour-suppressor inactivation.

Our data highlights the differences in mutational profile between localized intermediate risk cancers versus metastatic, castration-resistant prostate cancer (mCRPC). Nearly 50% of mCRPC tumours harbour mutations in AR, ETS genes, TP53 and PTEN and ˜20% have aberrations in DNA damage response genes (e.g. BRCA1, BRCA2, and ATM; which may portend sensitivity to PARP inhibitors²⁶⁻²⁸). Furthermore, more than 60% of mCRPC tumours contain clinically-actionable mutations that are non-AR related⁸. In contrast, non-SNV mutations dominate the genomics in localized non-indolent prostate cancer. No single gene was mutated at >10% frequency and the only prognostic SNV was ATM.

In the modern era of PSA screening, localized, non-indolent prostate cancer represents the vast majority of cases on initial clinical presentation. We show that localized disease represents a different biology from advanced mCRPC, which have undergone significant selective pressure, often through multiple courses of treatment²⁹. As recurrent SNV driver aberrations are rare in localized disease, tumours requiring intensified therapy may benefit from widespread genotoxic chemotherapy as supported by clinical trials in metastatic non-castrate disease³⁰. Similarly, the development of novel therapeutics will be improved by a robust understanding of the non-exomic drivers of aggression in localized prostate cancer.

Data Availability mRNA and methylation data is available in GEO under accession GSE84043. Raw sequencing data is available in EGA at: https://www.ebi.ac.uk/eqa/studies/EGAS00001000900. Processed variant calls have been uploaded to the ICGC Data Coordinating Centre. Baca and Barbieri WGS/WXS data is available on dbGaP under accession phs000447.v1.p1. Berger WGS data is available on dbGaP under accession phs000330.v1.p1. Weischenfeldt WGS data is available on EGA under accession EGAS00001000258. TCGA WGS/WXS data is available at Genomic Data Commons Data Portal under Project ID TCGA-PRAD.

### Copy Number Analysis

Prostate cancer may be a C-class tumour³¹. To investigate this postulate, we began by generating copy-number aberration (CNA) profiles of 284 localized prostate adenocarcinomas using OncoScan SNP arrays. For patients treated with image-guided radiotherapy (IGRT; n=147), a pre-treatment ultrasound-guided biopsy of the dominant lesion was taken and flash frozen. For radical prostatectomy (surgery) patients (n=137), the dominant lesion was excised from the gross specimen and flash frozen. All frozen tissue samples used for genomic studies contained at least 70% malignant epithelia prior to manual macro-dissection. Pathological Gleason scores for each sample analyzed were based on the consensus of two urological pathologists (data not shown).

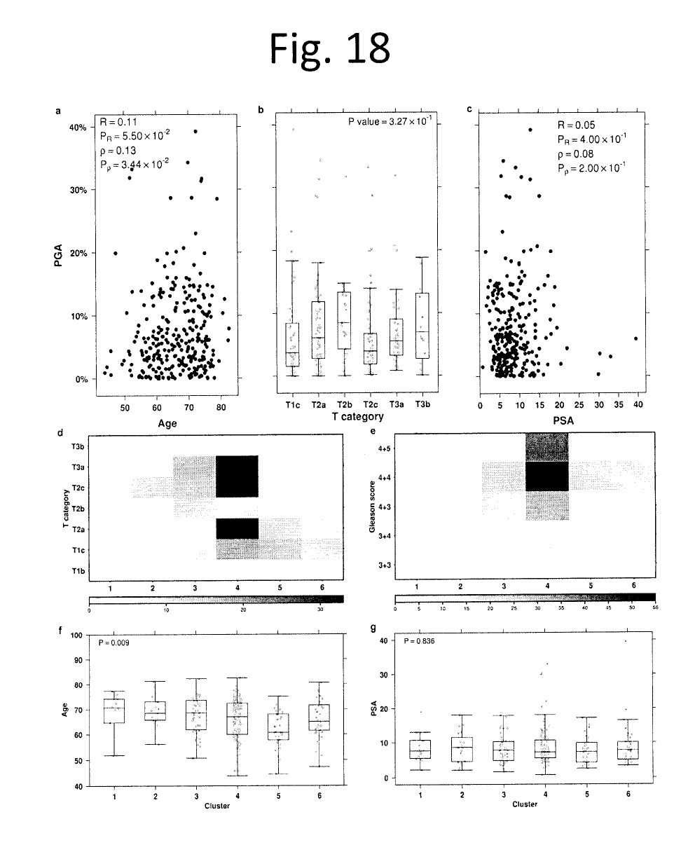

Our CNA analyses revealed a median of 34 separate CNAs encompassing 5.4% of the genome. We observed, however, dramatic inter-tumoural heterogeneity with tumours having between 0 and 267 CNAs, covering 0-39.2% of the genome (data not shown). Unsupervised analysis identified 6 CNA subtypes (FIG. 16a, 17a,b), which map closely with those previously identified in more heterogenous cohorts which contain higher Gleason scores³² (data not shown). We next compared global genomic instability, measured as the percentage of genome copy-number altered (PGA^{33,34}). PGA showed a clear association with Gleason score, with a median PGA of 2.2% for 3+3, 5.5% for 3+4, 8.8% for 4+3, 10.1% for 4+4 and 12.3% for 4+5 tumours (p=2.97×10⁻⁶; one-way ANOVA; FIG. 16b, 2c). The six CNA-based clusters closely associated with PGA—with clusters 4, 5 and 6 showing very low PGA relative to clusters 1, 2 and (p<1×10⁻¹⁶; one-way ANOVA). PGA was weakly associated with age (p=0.13, p=0.03), but not associated with T category (p=0.33; one-way ANOVA), or pre-treatment PSA (p=0.08; p=0.20; FIGS. 18a-c). By contrast, CNA clusters were not associated with age (p=0.009; one-way ANOVA), was associated with T category (p=2.83×10⁻⁵; Chi-sq), but not Gleason score (p=0.25; Chi-sq), or PSA (p=0.84; ANOVA; FIGS. 18d-g).

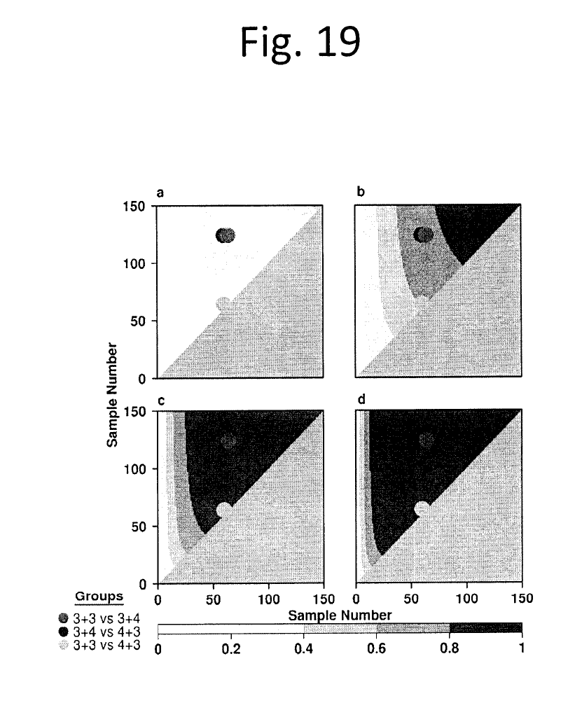

Despite this global difference in CNA-density, no single gene was mutated at statistically different frequencies amongst GS 3+3, 3+4 and 4+3 tumours (FIG. 16c and data not shown), despite sufficient statistical power (FIG. 19). GISTIC analysis identified 30 novel focal amplicons at a 5% FDR (15 deletions and 15 amplifications; FIG. 16d and data not shown). These include focal deletions in BRCA2 (33/284 samples) and NKX3-1 (146/284 samples), along with amplifications in region on chromosome 2p11.2 (chr2:89,116,398-89,292,829; 175/284 samples, no genes within it) and RET (86/284 samples). Several of these copy-number events were strongly associated with mRNA abundance changes, most notably KAT6A (deleted in 80/284 samples; p=0.002) and FANCA (deleted in 110/284 samples; p=0.004). We identified 539 genes whose CNA-status was associated with T category (FDR p<0.05), but no genes whose CNA-status was associated with serum PSA levels (data not shown). Genes associated with tumour-size included several well-known cancer driver genes, including PRDM16, CCND1, RPTOR1, TP73 and ERBB2.

TFBS Analyses of ncSNVs and Other Aberrations

To determine if these ncSNVs were biased to co-localizing in regulatory regions, we identified transcription-factor binding sites across the genome from the ENCODE project³⁵ and used binomial tests to assess enrichment of mutations in transcription factor binding sites (TFBS). We only considered genomic positions that were well-covered in our sequencing study (i.e. those with ≥10x coverage in normal and ≥17x in tumours) and assessed enrichment of 58 separate ChIP-Seq datasets. TFBSs enriched for mutations at a 5% FDR level included elevated mutation rate in H3K27 trimethylation sites, H3K9 trimethylation sites and H3K4 mono- and tri-methylation sites, all in multiple cell-lines and with multiple types of aberrations (FIG. 7c). The median tumour had 47 separate TFBSs enriched (range 1-58). Fascinatingly, androgen receptor binding-sites, as measured in two prostate cancer cell-lines (LNCaPs and PC3s) were strongly enriched for CNAs. The number of disrupted TFBSs was positively correlated with tumour size (R=0.15; p=0.012) and inversely correlated with patient age (R=−0.19; p=0.015), but not with GS, T2E status or pre-treatment PSA levels. Several individual TFBSs were correlated with specific clinical characteristics (data not shown). In particular, genomic rearrangements near androgen receptor binding-sites were inversely correlated with tumour size in both LNCaP and VCaP prostate cancer cells. These effects were independent of the size of the flanking region considered (FIG. 23).

### International Cancer Genome Consortium

The International Cancer Genome Consortium (ICGC) is a multi-national project aimed at comprehensively cataloging somatic mutations of at least 50 individual tumour-types by profiling the genomes of at least 500 tumours of each³⁶. This sample-size was selected to ensure sufficient statistical power to identify mutations present in 1% or more of individual patients. The present study from the Canadian Prostate Cancer Genome Network (CPC-GENE) provides a foundational piece towards achieving this goal, focusing on the genomes of localized GS6 and GS7 prostate cancer. This analysis of almost 500 exome-sequences provides near-saturation identification of genes altered by coding point-mutations. Moreover, the assessment of almost 200 whole-genomes provides an initial interrogation of recurrent genomic rearrangements and non-coding SNVs, representing—to our knowledge—the largest such study to date.

All documents disclosed herein, including those in the following reference list, are incorporated by reference. Although preferred embodiments of the invention have been described herein, it will be understood by those skilled in the art that that the detailed description and the specific examples while indicating preferred embodiments of the invention are given by way of illustration only, since various changes and modifications within the spirit and scope of the invention will become apparent to those skilled in the art from this detailed description.

### REFERENCES

1. Lozano, R. et al. Global and regional mortality from 235 causes of death for 20 age groups in 1990 and 2010: a systematic analysis for the Global Burden of Disease Study 2010. Lancet 380, 2095-2128, doi:10.1016/S0140-6736(12)61728-0 (2012).

2. Klotz, L. et al. Long-term follow-up of a large active surveillance cohort of patients with prostate cancer. J Clin Oncol 33, 272-277, doi:10.1200/JCO.2014.55.1192 (2015).

3 D'Amico, A. V. et al. Cancer-specific mortality after surgery or radiation for patients with clinically localized prostate cancer managed during the prostate-specific antigen era. J Clin Oncol 21, 2163-2172 (2003).

4. Boutros, P. C. et al. Spatial genomic heterogeneity within localized, multifocal prostate cancer. Nature Genetics 47, 736-745, doi:10.1038/ng.3315 (2015).

5. Cooper, C. S. et al. Analysis of the genetic phylogeny of multifocal prostate cancer identifies multiple independent clonal expansions in neoplastic and morphologically normal prostate tissue. Nat. Genet. 47, 367-372, doi:10.1038/ng.3221 (2015).

6. Lalonde, E. et al. Tumour genomic and microenvironmental heterogeneity for integrated prediction of 5-year biochemical recurrence of prostate cancer: a retrospective cohort study. Lancet Oncol 15, 1521-1532, doi:10.1016/S1470-2045(14)71021-6 (2014).

7. Buyyounouski, M. K., Pickles, T., Kestin, L. L., Allison, R. & Williams, S. G. Validating the interval to biochemical failure for the identification of potentially lethal prostate cancer. J Clin Oncol 30, 1857-1863, doi:10.1200/JCO.2011.35.1924 (2012).

8. Robinson, D. et al. Integrative clinical genomics of advanced prostate cancer. Cell 161, 1215-1228, doi:10.1016/j.ce11.2015.05.001 (2015).

9. Berger, M. F. et al. The genomic complexity of primary human prostate cancer. Nature 470, 214-220, doi:10.1038/nature09744 (2011).

10. Baca, S. C. et al. Punctuated evolution of prostate cancer genomes. Cell 153, 666-677, doi:10.1016/j.ce11.2013.03.021 (2013).

11. Weischenfeldt, J. et al. Integrative genomic analyses reveal an androgen-driven somatic alteration landscape in early-onset prostate cancer. Cancer Cell 23, 159-170, doi:10.1016/j.ccr.2013.01.002 (2013).

12. Cancer Genome Atlas Research Network. The Molecular Taxonomy of Primary Prostate Cancer. Cell 163, 1011-1025, doi:10.1016/j.cell.2015.10.025 (2015).

13. Barbieri, C. E. et al. Exome sequencing identifies recurrent SPOP, FOXA1 and MED12 mutations in prostate cancer. Nat. Genet. 44, 685-689, doi:10.1038/ng.2279 (2012).

14. Ewing, A. D. et al. Combining tumor genome simulation with crowdsourcing to benchmark somatic single-nucleotide-variant detection. Nat Methods 12, 623-630, doi:10.1038/nmeth.3407 (2015).

15. Lawrence, M. S. et al. Discovery and saturation analysis of cancer genes across 21 tumour types. Nature 505, 495-501, doi:10.1038/nature12912 (2014).

16. Ciriello, G. et al. Emerging landscape of oncogenic signatures across human cancers. Nat. Genet. 45, 1127-1133, doi:10.1038/ng.2762 (2013).

17. Zhou, J. & Troyanskaya, O. G. Predicting effects of noncoding variants with deep learning-based sequence model. Nat Methods 12, 931-934, doi:10.1038/nmeth.3547 (2015).

18. Alexandrov, L. B. et al. Signatures of mutational processes in human cancer. Nature 500, 415-421, doi:10.1038/nature12477 (2013).

19. Gandhi, M., Evdokimova, V. & Nikiforov, Y. E. Frequency of close positioning of chromosomal loci detected by FRET correlates with their participation in carcinogenic rearrangements in human cells. Genes, chromosomes & cancer 51, 1037-1044, doi:10.1002/gcc.21988 (2012).

20. Nikiforova, M. N. et al. Proximity of chromosomal loci that participate in radiation-induced rearrangements in human cells. Science 290, 138-141 (2000).

21. Rickman, D. S. et al. Oncogene-mediated alterations in chromatin conformation. Proc. Natl. Acad. Sci. U.S.A. 109, 9083-9088, doi:10.1073/pnas.1112570109 (2012).

22. Korbel, J. O. & Campbell, P. J. Criteria for inference of chromothripsis in cancer genomes. Cell 152, 1226-1236, doi:10.1016/j.ce11.2013.02.023 (2013).

23. Govind, S. K. et al. ShatterProof: operational detection and quantification of chromothripsis. BMC Bioinfo. 15, 78, doi:10.1186/1471-2105-15-78 (2014).

24. Zafarana, G. et al. Copy number alterations of c-MYC and PTEN are prognostic factors for relapse after prostate cancer radiotherapy. Cancer 118, 4053-4062, doi:10.1002/cncr.26729 (2012).

25. Hieronymus, H. et al. Copy number alteration burden predicts prostate cancer relapse. Proceedings of the National Academy of Sciences of the United States of America 111, 11139-11144, doi:10.1073/pnas.1411446111 (2014).

26. Mateo, J. et al. DNA-Repair Defects and Olaparib in Metastatic Prostate Cancer. The New England journal of medicine 373, 1697-1708, doi:10.1056/NEJMoa1506859 (2015).

27. Schiewer, M. J. et al. Dual roles of PARP-1 promote cancer growth and progression. Cancer discovery 2, 1134-1149, doi:10.1158/2159-8290.CD-12-0120 (2012).

28. Feng, F. Y., de Bono, J. S., Rubin, M. A. & Knudsen, K. E. Chromatin to Clinic: The Molecular Rationale for PARP1 Inhibitor Function. Molecular cell 58, 925-934, doi:10.1016/j.molcel.2015.04.016 (2015).

29. Gundem, G. et al. The evolutionary history of lethal metastatic prostate cancer. Nature 520, 353-357, doi:10.1038/nature14347 (2015).

30. Graff, J. N. & Beer, T. M. Should docetaxel be administered earlier in prostate cancer therapy? Expert Rev Anticancer Ther 15, 977-979, doi:10.1586/14737140.2015.1074042 (2015).

31. Ciriello, G. et al. Emerging landscape of oncogenic signatures across human cancers. Nat. Genet. 45, 1127-1133, doi:10.1038/ng.2762 (2013).

32. Taylor, B. S. et al. Integrative genomic profiling of human prostate cancer. Cancer Cell 18, 11-22, doi:10.1016/j.ccr.2010.05.026 (2010).

33. Lalonde, E. et al. Tumour genomic and microenvironmental heterogeneity for integrated prediction of 5-year biochemical recurrence of prostate cancer: a retrospective cohort study. Lancet Oncol 15, 1521-1532, doi:10.1016/S1470-2045(14)71021-6 (2014).

34. Hieronymus, H. et al. Copy number alteration burden predicts prostate cancer relapse. Proceedings of the National Academy of Sciences of the United States of America 111, 11139-11144, doi:10.1073/pnas.1411446111 (2014).

35. Encode Project Consortium. An integrated encyclopedia of DNA elements in the human genome. Nature 489, 57-74, doi:10.1038/nature11247 (2012).

36. Hudson, T. J. et al. International network of cancer genome projects. Nature 464, 993-998, doi:nature08987 [pii] 10.1038/nature08987 (2010).

37. Van Loo, P. et al. Analyzing cancer samples with SNP arrays. Methods in molecular biology 802, 57-72, doi:10.1007/978-1-61779-400-1_4 (2012).

38. Song, S. et al. qpure: A tool to estimate tumor cellularity from genome-wide single-nucleotide polymorphism profiles. PLoS One 7, e45835, doi:10.1371/journal.pone.0045835 (2012).

39. Alexandrov, L. B. et al. Signatures of mutational processes in human cancer. Nature 500, 415-421, doi:10.1038/nature12477 (2013).

1. A method of prognosing and/or predicting disease progression and/or in subject with prostate cancer, the method comprising:
a) providing a sample containing genetic material from cancer cells;
b) determining or measuring at least 2 patient biomarkers regarding the prostate cancer tumor selected from the group consisting of: T category, ACTL6B methylation, TCERGL1 methylation, chr7:61 Mbp inter-chromosomal translocation, ATM single nucleotide variants and MYC copy number aberrations;
c) comparing said patient biomarkers to corresponding reference or control biomarkers; and
d) determining the likelihood of disease progression;
wherein a likelihood of disease progression is higher with each of ACTL6B hyper-methylation, TCERGL1 hypo-methylation, higher T category, and higher incidences of chr7:61 Mbp inter-chromosomal translocation, ATM single nucleotide variants and MYC copy number aberrations, when the difference is statistically significant on comparison with the reference or control biomarkers.

2. The method according to of claim 1, wherein the at least 2 patient biomarkers, is at least 3, 4, 5 or 6 patient biomarkers.

3. The method of claim 1, where the prostate cancer is localized prostate cancer, preferably non-indolent localized prostate cancer.

4. The method of claim 1, further comprising building a patient biomarker profile from the determined or measured patient biomarkers.

5. The method of claim 1, wherein the prediction of disease progression is following at least one of active surveillance, surgery, endocrine therapy, chemotherapy, radiotherapy, hormone therapy, gene therapy, thermal therapy, and ultrasound therapy.

6. The method of claim 1, further comprising classifying the patient into a high risk group if the likelihood of disease progression is relatively high or a low risk group if the likelihood of disease progression is relatively low.

7. The method of claim 6, further comprising treating the patient with more aggressive therapy if the patient is in the high risk group.

8. The method of claim 7, wherein the more aggressive therapy comprises adjuvant therapy, preferably hormone therapy, chemotherapy or radiotherapy.

9. A computer-implemented method of predicting disease progression in patient with prostate cancer, the method comprising:
a) receiving, at at least one processor, data reflecting at least 2 patient biomarkers regarding the prostate cancer tumor selected from the group consisting of: T category, ACTL6B methylation, TCERGL1 methylation, chr7:61 Mbp inter-chromosomal translocation, ATM single nucleotide variants and MYC copy number aberrations;
b) constructing, at the at least one processor, an expression profile corresponding to the expression levels;
c) comparing, at the at least one processor, said patient biomarkers to corresponding reference or control biomarkers;
d) determining, at the at least one processor, the likelihood of disease progression;
wherein a likelihood of disease progression is higher with each of ACTL6B hyper-methylation, TCERGL1 hypo-methylation, higher T category, and higher incidences of chr7:61 Mbp inter-chromosomal translocation, ATM single nucleotide variants and MYC copy number aberrations, when the difference is statistically significant on comparison with the reference or control biomarkers.

10. The method of claim 9, wherein the at least 2 patient biomarkers, is at least 3, 4, 5 or 6 patient biomarkers.

11. A computer program product for use in conjunction with a general-purpose computer having a processor and a memory connected to the processor, the computer program product comprising a computer readable storage medium having a computer mechanism encoded thereon, wherein the computer program mechanism may be loaded into the memory of the computer and cause the computer to carry out the method of claim 1.

12. A computer readable medium having stored thereon a data structure for storing the computer program product of claim 11.

13. A device for predicting disease progression in patient with prostate cancer, the device comprising:
at least one processor; and
electronic memory in communication with the at least one processor, the electronic memory storing processor-executable code that, when executed at the at least one processor, causes the at least one processor to:
a) receive data reflecting at least 2 patient biomarkers regarding the prostate cancer tumor selected from the group consisting of: T category, ACTL6B methylation, TCERGL1 methylation, chr7:61 Mbp inter-chromosomal translocation, ATM single nucleotide variants and MYC copy number aberrations;
b) compare said patient biomarkers to corresponding reference or control biomarkers; and
c) determine, at the at least one processor, the likelihood of disease progression;
wherein a likelihood of disease progression is higher with each of ACTL6B hyper-methylation, TCERGL1 hypo-niethylation, higher T category, and higher incidences of chr7:61 Mbp inter-chromosomal translocation, ATM single nucleotide variants and MYC copy number aberrations, when the difference is statistically significant on comparison with the reference or control biomarkers.

14. The device of claim 13, wherein the at least 2 patient biomarkers, is at least 3, 4, 5 or 6 patient biomarkers.
